(19) United States
(12) Patent Application Publication
(10) Pub. No.: US 2026/0078100 A1
(43) Pub. Date: Mar. 19, 2026

(54) PROCESS FOR REDUCING THE OVERCHLORIDING OF HIGH SELECTIVITY ETHYLENE OXIDE CATALYSTS DURING RESTARTS

(71) Applicant: Dow Global Technologies LLC, Midland, MI (US)

(72) Inventors: Liping Zhang, Lake Jackson, TX (US); Albert C. Liu, Charleston, WV (US)

(21) Appl. No.: 19/106,369

(22) PCT Filed: Oct. 3, 2023
(86) PCT No.: PCT/US2023/075768

§ 371 Date: Feb. 25, 2025

Related U.S. Application Data
(60) Provisional application No. 63/412,919, filed on Oct. 4, 2022.

(51) Int. Cl. C07D 301/10

(52) U.S. Cl. CPC C07D 301/10

(57) ABSTRACT

Disclosed herein are methods of improving the restart performance of a high-selectivity ethylene oxide catalyst that has been shut down due to a planned or unplanned outage. It is believed that the proper selection of process variables on restart avoids exacerbating catalyst surface overchloriding that may occur when subsurface chlorides are diffused to the catalyst surface. In accordance with the method, the initial restart reaction temperature is no more than five (5) degrees Celsius above an initial start-up steady-state reaction temperature on fresh catalyst and is no more than five (5) degrees Celsius below the initial start-up steady-state reaction temperature. The addition of a make-up organic chloride to the reactor feed gas is deferred until the reactor outlet oxygen concentration equals or exceeds 0.5 mole percent and until a relative scaled reactor vinyl chloride feed gas concentration is no greater than 110 percent.

### TECHNICAL FIELD

This disclosure relates generally to processes for making ethylene oxide, and more specifically, to a method of reducing the extent of overchloriding high-selectivity ethylene oxide catalysts when restarting the process.

### BACKGROUND

This disclosure relates to a process for manufacturing ethylene oxide (EO). Ethylene oxide is used to produce ethylene glycol, which is used as an automotive coolant, as antifreeze, and in preparing polyester fibers and resins, nonionic surfactants, glycol ethers, ethanolamines, and polyethylene polyether polyols.

The production of ethylene oxide generally occurs via the catalytic epoxidation of ethylene in the presence of oxygen. Along with ethylene and oxygen, gas phase promoters, such as certain organic chloride compounds, are provided in the reactor feed gas and deposit promoting species on the surface of the catalyst in order to enhance the selectivity and/or activity for the production of ethylene oxide.

Conventional silver-based catalysts used in such processes provide a relatively low efficiency or “selectivity” (i.e., a lower percentage of the reacted ethylene is converted to the desired ethylene oxide). In certain exemplary processes, when using conventional catalysts in the epoxidation of ethylene, the theoretically maximal selectivity towards ethylene oxide, expressed as a fraction of the ethylene converted, does not reach values above the 6/7 or 85.7 percent limit. Therefore, this limit had long been considered to be the theoretically maximal selectivity of this reaction, based on the stoichiometry of the following reaction equation:

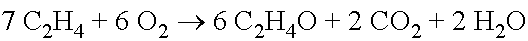

cf. the Kirk-Othmer Encyclopedia of Chemical Technology, 4th ed., Vol. No. 9, 1994, p. 926.

Certain “high efficiency” or “high selectivity” silver-based catalysts are highly selective towards ethylene oxide production. For example, when using certain catalysts in the epoxidation of ethylene, the theoretically maximal selectivity towards ethylene oxide can reach values above the 6/7 or 85.7 percent limit referred to, for example 88 percent, or 89 percent, or above. High selectivity catalysts comprise as their active components silver, rhenium, and at least one further metal. See EP0352850B1 and WO2007/123932.

Conventional catalysts have relatively flat selectivity curves with respect to the gas phase promoter concentration in the feed, i.e., the selectivity is almost invariant (i.e., the change in selectivity with respect to a change in gas phase promoter concentration in the feed is less than about 0.1%/ppmv) over a wide range of such promoter concentrations, and this invariance is substantially unaltered as reaction temperature is changed during prolonged operation of the catalyst. However, conventional catalysts have nearly linear activity decline curves with respect to the gas phase promoter concentration in the feed, i.e., with increasing gas phase promoter concentration in the feed, temperature has to be increased or the ethylene oxide production rate will be reduced. Therefore, when using a conventional catalyst, for optimum selectivity, the gas phase promoter concentration in the feed can be chosen at a level at which the maximum selectivity can be maintained at relatively low operating temperatures. For conventional catalysts, the reaction temperature may be adjusted to obtain a desired production rate without any substantial need to adjust the gas phase promoter concentration.

By contrast, high selectivity catalysts tend to exhibit relatively steep selectivity curves as a function of gas phase promoter concentration as the concentration moves away from the value that provides the highest selectivity (i.e., the change in selectivity with respect to a change in gas phase promoter concentration is at least about 0.2%/ppmv when operating away from the selectivity maximizing promoter concentration). Thus, small changes in the promoter concentration can result in significant selectivity changes, and the selectivity exhibits a pronounced maximum, i.e., an optimum, at certain concentrations (or feed rates) of the gas phase promoter, when reactor pressure and feed gas composition are kept unchanged for a given reaction temperature and catalyst age.

It has been found that when restarting ethylene oxide processes that use chloride compounds as gas phase promoters with high selectivity, rhenium-promoted silver catalysts, the performance on restart is often poorer than expected, making it difficult to return to stable operation and maximum selectivity for a given target value of an ethylene oxide production parameter.

For commercial EO processes, due to the full gas recycle design, excess chloride compounds in the gas phase resulting from surface dechlorination during restarts cannot be removed quickly. The greater than desired gas phase chloride concentrations result in greater than desired catalyst surface chloriding. Without wishing to be bound by any theory, at least in some cases, it is believed that upon restart after being shut down for periods of time of several hours or more, high-efficiency ethylene oxide catalysts can desorb and release subsurface chloride compounds that have migrated to the surface, which are then re-deposited on the surface of the catalyst from the gas phase, often resulting in greater than desired catalyst surface chloriding and prolonged periods of low selectivity towards the production of ethylene oxide. Whether this particular problem is encountered during any particular restart is believed to depend on the conditions to which the catalyst is exposed during the off-line period. Also, the selection of process variable values and the manner in which they are adjusted upon restart strongly influences the extent of surface chlorination over the course of the restart.

Thus, a need has arisen for a method of improving the restart performance of a high-selectivity, rhenium-promoted silver ethylene oxide catalyst.

### BRIEF DESCRIPTION OF THE FIGURES

FIG. 1 is a process flow diagram depicting an embodiment of a process for making ethylene oxide by epoxidizing ethylene over a high selectivity, rhenium-promoted silver catalyst;

FIGS. 2A-2C are flow diagrams describing methods of restarting a high-efficiency, ethylene oxide process following an off-line period;

FIGS. 3A-3I are plots of efficiency, relative scaled vinyl chloride feed gas concentration, reactor inlet carbon dioxide concentration, reactor coolant temperature, relative feed gas ethyl chloride concentration, reactor outlet carbon dioxide concentration, relative ethylene oxide production rate, relative make-up oxygen feed rate, and relative make-up ethyl chloride feed rate to make-up oxygen feed rate ratio versus ethylene oxide (EO) production time for a batch of catalyst in a first commercial scale high-efficiency, ethylene oxide process restarted in accordance with the present disclosure;

FIGS. 4A-4I are plots of efficiency, relative scaled vinyl chloride feed gas concentration, reactor inlet carbon dioxide concentration, reactor coolant temperature, relative feed gas ethyl chloride concentration, reactor outlet carbon dioxide concentration, relative ethylene oxide production rate, relative make-up oxygen feed rate, and relative make-up ethyl chloride feed rate to make-up oxygen feed rate ratio versus ethylene oxide (EO) production time for a first comparative example in which the batch of catalyst in the first commercial scale high-efficiency, ethylene oxide process of FIGS. 3A-3I was not restarted in accordance with the present disclosure;

FIGS. 5A-5I are plots of efficiency, relative scaled vinyl chloride feed gas concentration, reactor inlet carbon dioxide concentration, reactor coolant temperature, relative feed gas ethyl chloride concentration, reactor outlet carbon dioxide concentration, relative ethylene oxide production rate, relative make-up oxygen feed rate, and relative make-up ethyl chloride feed rate to make-up oxygen feed rate ratio versus ethylene oxide (EO) production time for a second comparative example in which a batch of catalyst in a pilot plant high-efficiency, ethylene oxide process was not restarted in accordance with the present disclosure;

FIGS. 6A-6I are plots of efficiency, relative scaled vinyl chloride feed gas concentration, reactor inlet carbon dioxide concentration, reactor coolant temperature, relative feed gas ethyl chloride concentration, reactor outlet carbon dioxide concentration, relative ethylene oxide production rate, relative make-up oxygen feed rate, and relative make-up ethyl chloride feed rate to oxygen feed rate ratio versus ethylene oxide (EO) production time for an example in which the batch of catalyst in the pilot plant high-efficiency, ethylene oxide process of the second comparative example was restarted in accordance with the present disclosure; and

FIGS. 7A-7I are plots of efficiency, relative scaled vinyl chloride feed gas concentration, reactor inlet carbon dioxide concentration, reactor coolant temperature, relative feed gas ethyl chloride concentration, reactor outlet carbon dioxide concentration, relative ethylene oxide production rate, relative make-up oxygen feed rate, and relative make-up ethyl chloride feed rate to make-up oxygen feed rate ratio versus ethylene oxide (EO) production time for a third comparative example in which a batch of catalyst in a second commercial scale high-efficiency, ethylene oxide process was not restarted in in accordance with the present disclosure.

### DETAILED DESCRIPTION

The present disclosure provides methods of restarting a process for producing ethylene oxide by reacting ethylene and oxygen in the presence of at least one organic chloride over a batch of high-efficiency, rhenium-promoted silver catalyst. The method is performed following a period during which the process is off-line (i.e., shutdown) and no make-up oxygen is fed to the batch of catalyst. The period of time is preferably at least about two hours, more preferably at least about three hours, and still more preferably at least about 4 hours. Upon restart, a feed gas comprising a reactor recycle stream combined with make-up streams comprising at least make-up ethylene at an initial feed rate of make-up ethylene and make-up oxygen at an initial feed rate of make-up oxygen is fed to the batch of high-efficiency catalyst, and the reaction temperature is adjusted to an initial restart reaction temperature value that is no more than five (5), preferably no more than three (3) and more preferably no more than two (2) degrees Celsius above the initial steady-state reaction temperature achieved for the catalyst batch when the batch was fresh (i.e., “the initial start-up steady-state reaction temperature value”). At the same time, the initial restart reaction temperature value is no more than five (5), preferably no more than three (3), and more preferably not more than two (2) degrees Celsius below the initial steady-state reaction temperature value achieved when the batch of catalyst was fresh. In preferred examples, the initial restart reaction temperature value is no greater than the pre-shutdown steady-state temperature value. It is also preferred that the pre-shutdown steady state reaction temperature value is more than five (5), more than ten (10), or more than fifteen (15) degrees Celsius higher than the initial start-up steady-state reaction temperature value.

In accordance with the method, no make-up organic chloride is fed to the batch of catalyst until the reactor outlet oxygen concentration exceeds a value of 0.5 mole percent, preferably 0.7 mole percent, and more preferably 0.9 mole percent. When the reactor outlet concentration of oxygen exceeds a value of 0.5 mole percent, preferably 0.7 mole percent, and more preferably 0.9 mole percent, a make-up organic chloride selected from ethyl chloride and ethylene dichloride is fed to the batch of high-efficiency catalyst at a rate that is no greater than 30 percent, preferably no greater than 25 percent, and more preferably no greater than 20 percent of the last steady state make-up organic chloride feed rate value that preceded the period during which the reactor was off-line.

In additional preferred examples, depending on the value of the selectivity(S) versus the pre-shutdown, steady-state selectivity (Sₛₛ), and the direction in which the selectivity is trending, the reaction temperature and feed rate of make-up oxygen are slowly ramped up to their pre-shutdown steady-state values (T_{Rx SS}, F_{O2 MU SS}). In the same or other examples, depending on the value of the selectivity(S) versus the steady-state, pre-shutdown selectivity (Sₛₛ), the direction in which the selectivity is trending, a value of a relative scaled reactor feed gas vinyl chloride (VCI) concentration (C_{VCI RS}), and the direction in which the relative scaled reactor feed gas vinyl chloride concentration is trending, the feed rate of make-up organic chloride is adjusted to achieve and maintain a ratio of feed gas make-up organic chloride feed rate to feed gas make-up oxygen feed rate that is no more than 115 percent of, preferably no more than 105 percent of, and more preferably no more than 100 percent of the ratio of the respective last steady-state feed rates that preceded the shut-down.

The present specification provides certain definitions to guide those of ordinary skill in the art in the practice of the present invention. Provision, or lack of provision, of a definition for a particular term or phrase is not meant to imply any particular importance, or lack thereof; rather, and unless otherwise noted, terms are to be understood according to the conventional usage by those of ordinary skill in the relevant art. Unless defined otherwise, technical and scientific terms used herein have the same meaning as commonly understood by one of skill in the art to which this invention belongs.

The “activity” of a catalyst in a fixed bed reactor is generally defined as the reaction rate towards the desired product per unit of catalyst volume in the reactor. The activity of a catalyst can be quantified in a number of ways, one being the mole percent of ethylene oxide contained in the outlet stream of the reactor relative to that in the inlet stream (the mole percent of ethylene oxide in the inlet stream typically, but not necessarily, approaches zero percent) while the reaction temperature is maintained substantially constant; and another being the temperature required to maintain a given rate of ethylene oxide production. In many instances, activity is measured over a period of time in terms of the mole percent of ethylene oxide produced at a specified constant temperature. Alternatively, activity may be measured as a function of the temperature used to sustain production of a specified constant mole percent of ethylene oxide.

The “efficiency” of the oxidation, which is synonymous with “selectivity,” refers to the relative amount (as a fraction or in percent) of converted or reacted ethylene that forms a particular product. For example, the “selectivity to ethylene oxide” refers to the percentage on a molar basis of converted ethylene that forms ethylene oxide.

The term “ethylene oxide production parameter” is used herein to describe a variable that relates to the extent to which ethylene oxide is produced. Examples of ethylene oxide production parameters include ethylene oxide concentration, ethylene oxide yield, ethylene oxide production rate, ethylene oxide production rate/catalyst bed volume, ethylene conversion, oxygen conversion, oxygen feed rate, and ethylene feed rate. Thus, the ethylene oxide concentration relates to the ethylene oxide production rate because the production rate may be obtained by multiplying the ethylene oxide concentration and the net product flow rate from the reactor. The ethylene oxide production rate/catalyst bed volume may be determined by dividing the production rate by the volume of the catalyst bed. The oxygen and ethylene conversions, as well as the oxygen and ethylene feed rates at steady-state conditions, are related to the production of the ethylene oxide by the selectivity. Selectivity and activity are not ethylene oxide production parameters. A “target ethylene oxide production parameter” is an ethylene oxide production parameter that is used as a specification for operating an ethylene oxide process. In one example, an ethylene oxide process is operated to achieve a specified value of an ethylene oxide production rate, in which case the ethylene oxide production rate would be considered a target ethylene oxide production parameter.

“Gas phase promoters” means compounds that enhance the selectivity and/or activity of a process for the production of ethylene oxide. Preferred gas phase promoters include organic chlorides. More preferably, the gas phase promoter is at least one selected from the group consisting of methyl chloride, ethyl chloride, ethylene dichloride, vinyl chloride, and mixtures thereof. Ethyl chloride and ethylene dichloride are most preferred as the gas phase promoter fed into the process.

The terms “high efficiency catalyst” and “high selectivity catalyst” refer to a catalyst that is capable of producing ethylene oxide from the ethylene and oxygen at a selectivity greater than 85.7 percent. The observed actual selectivity of a high selectivity catalyst may fall below 85.7 percent under certain conditions based on process variables, catalyst age, and the like. However, if the catalyst is capable of achieving at least an 85.7 percent selectivity, at any point during its life, for example, under any set of reaction conditions, or by extrapolating lower efficiencies observed at two different oxygen conversions obtained by varying gas hourly space velocity to the limiting case of zero oxygen conversion, it is considered to be a high selectivity catalyst.

The term “oxygen flammability concentration” refers to a concentration of oxygen based on the oxygen flammability limit and includes both the oxygen flammability limit itself and oxygen concentrations that are biased from the oxygen flammability limit by a safety margin.

“Reaction temperature,” or “(T_{Rx})” refers to any selected temperature(s) that are directly or indirectly indicative of the catalyst bed temperature. In certain embodiments, the reaction temperature may be a catalyst bed temperature at a specific location in the catalyst bed. In other embodiments, the reaction temperature may be a numerical average of several catalyst bed temperature measurements made along one or more catalyst bed dimensions (e.g., along the length). In additional embodiments, the reaction temperature may be the reactor outlet gas temperature. In further embodiments, the reaction temperature may be a reactor coolant temperature selected from the reactor coolant outlet temperature, the reactor coolant inlet temperature, for example, measured in the reactor shell, and a reactor coolant temperature along the tube length between the coolant inlet and outlet, such as at the mid-point of the tube length. In the case of a boiling coolant reactor, the temperature may be determined from the coolant pressure.

The term “initial start-up steady-state reaction temperature value” (T_{Rx i SS}) is the first steady-state reaction temperature value achieved after a fresh high-efficiency ethylene oxide catalyst is subjected to a reactive mixture of ethylene and oxygen.

The term “initial restart reaction temperature value” refers to the reaction temperature of a high-efficiency ethylene oxide process at the time of initiating make-up oxygen feed during a restart.

The terms “minimum controllable organic chloride feed rate” (F_{RCI MU min}), “minimum controllable ethyl chloride feed rate” (F_{ECI MU min}), and “minimum controllable ethylene dichloride feed rate” (F_{EDC MU min}) refer to the smallest, stable non-zero gas flow rate of all make-up organic chloride streams (collectively), a make-up ethyl chloride stream, or a make-up ethylene dichloride stream, respectively.

The term “overchloriding,” sometimes also referred to as “overmoderating” in the prior art, refers to a process by which the chloride promoter concentration on the surface of a high selectivity ethylene oxide catalyst is caused to be in excess of an optimal surface concentration that yields the maximum selectivity under a given set of operating conditions. Adjustment of the catalyst surface chloride concentration is typically accomplished by varying the gas-phase concentration of one or more organic chlorides in the feed to a reactor by adjusting the feed rate of the make-up organic chloride into the recycle stream.

“Shutdown” or “off-line” refers to a period of time during which a high efficiency catalyst is not subjected to a reactive mixture of oxygen and ethylene. A “trip” refers to a shutdown that occurs due to a process upset or some unexpected occurrence, such as a pump failure, a recycle gas compressor failure, a downstream shutdown, a disruption in the feed, a failure of control valves that regulate pressures, temperatures or flow rates, etc.

“Ethylene oxide production time”, “ethylene oxide production days” or “ethylene oxide production hours” means, respectively, a duration of time, a number of days, and a number of hours during which ethylene oxide is produced by an ethylene oxide process, measured cumulatively from the initial start-up of a batch of fresh silver catalyst.

“Ramp”, “Ramping”, or “Ramp Rate” when used to refer to changes in the value of a process variable over a period of time are not limited to the slopes of continuous linear adjustments but can also include non-continuous and/or non-linear adjustments such as step changes divided by a period of time over which such adjustments are made.

“Relative feed rate of make-up ethylene” or “Relative make-up ethylene feed rate” means a current value of the make-up ethylene feed rate (F_{Et=MU}) divided by the pre-shutdown, steady-state value of the make-up ethylene feed rate (F_{Et=MU SS}), expressed as a percentage or a fraction.

“Relative feed rate of make-up ethyl chloride” or “Relative make-up ethyl chloride feed rate” refers to the molar or volumetric flow rate of make-up ethyl chloride following a restart divided by the pre-shutdown, steady-state molar or volumetric flow rate of make-up ethyl chloride.

“Relative feed rate of make-up ethylene dichloride or “Relative make-up ethylene dichloride feed rate” refers to the molar or volumetric flow rate of make-up ethylene dichloride following a restart divided by the pre-shutdown, steady-state molar or volumetric flow rate of make-up ethylene dichloride.

“Relative feed rate of make-up oxygen” or “Relative make-up oxygen feed rate” means a current value of the make-up oxygen feed rate (F_{O2 MU}) divided by the pre-shutdown, steady-state make-up oxygen feed rate (F_{O2 MU SS}), expressed as a percentage or a fraction.

“Relative EC1/O₂ feed ratio” or “relative ethyl chloride to oxygen feed ratio” means the ratio of a first, current ratio of the make-up ethyl chloride feed rate value to the make-up oxygen feed rate value (F_{ECI MU})/(F_{O2 MU}) to a second ratio of the pre-shutdown, steady-state make-up ethyl chloride feed rate value to the pre-shutdown, steady-state make-up oxygen feed rate value (F_{ECL MU SS})/(F_{O2 MU SS}), expressed as a percentage or a fraction. An analogous “Relative EDC/O₂ feed ratio” or “relative ethylene dichloride to oxygen feed ratio” may be defined when the make-up organic chloride is ethylene dichloride.

“Relative Ethylene Oxide Production Parameter Value” or “Relative EO Production Parameter Value” means the ratio of a current value of an ethylene oxide production parameter (EO_{PP}) to a pre-shutdown, steady-state value of the ethylene oxide production parameter (EO_{PP SS}), expressed as a percentage or a fraction.

“Relative EO Production Rate” means the ratio of a current value of the EO production rate to the pre-shutdown, steady-state value of the EO production rate (EO_{Prod SS}).

“Steady-State” when referring to an ethylene oxide process refers to an ethylene oxide production process in which (i) a value of at least one ethylene oxide production parameter has achieved a daily average value that fluctuates from a target value of the at least one ethylene oxide production parameter by no more than two (2) percent for a period of at least two (2), preferably five (5), and more preferably seven (7) days, and (ii) a value of the efficiency has achieved a daily average value that fluctuates by no more than 0.2 percentage points from the value averaged over the same period.

“Steady-State” when referring the value of a process variable refers to an average value over a corresponding steady-state period for the ethylene oxide process;

“Pre-shutdown, steady-state” when referring to a value of a particular process variable means the average value of the variable over the last steady-state period before a shutdown preceding a restart of an ethylene oxide process.”

High selectivity silver-based catalysts comprising rhenium and methods of making them are known to those of skill in the art. See EP0352850B1, WO2007/123932, WO2014/150669, EP1613428, or CN102133544.

Suitable reactors for the epoxidation reaction include fixed bed reactors, fixed bed tubular reactors, continuous stirred tank reactors (CSTR), fluid bed reactors and a wide variety of reactors that are well known to those skilled in the art. The desirability of recycling unreacted feed, or employing a single-pass system, or using successive reactions to increase ethylene conversion by employing reactors in series arrangement can also be readily determined by those skilled in the art. However, the problems addressed by the present disclosure are particularly severe when at least a portion of the reactor outlet stream is recycled to form part of the feed gas to the reactor. The epoxidation reaction is carried out at a temperature that is preferably at least about 200° C., more preferably at least about 210° C., and most preferably at least about 220° C. Reaction temperatures of no more than about 300° C. are preferred, more preferably not more than about 290° C., and most preferably not more than about 280° C.

The reactor pressure is selected based on the desired mass velocity and productivity and ranges generally from about 5 atm (506 kPa) to about 30 atm (3.0 MPa). The gas hourly space velocity (GHSV) is preferably greater than about 3,000 hr⁻¹, more preferably greater than about 4,000 hr⁻¹, and most preferably greater than about 5,000 hr⁻¹.

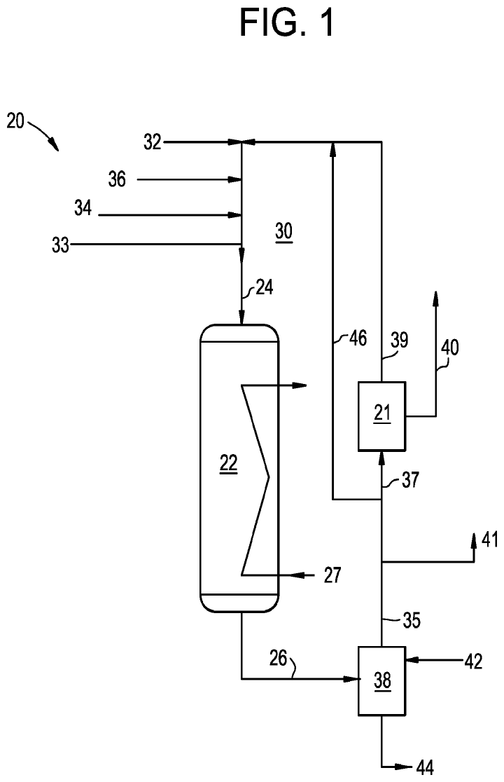

FIG. 1 is a process flow diagram depicting an embodiment of a process 20 for making ethylene oxide by epoxidizing ethylene over a high selectivity, rhenium-promoted silver catalyst. Process 20 includes a reactor 22 comprising multiple reactor tubes with a high selectivity catalyst therein. Ethylene make-up feed stream 36 (which may also include saturated hydrocarbons, such as ethane as an impurity), ballast gas 32, oxygen make-up feed 34, and gas phase promoter make-up feed 33 each combine with recycle stream 30 to yield reactor feed gas inlet stream 24 proximate to the reactor 22. The reactor product stream 26 includes the ethylene oxide product in addition to side products (e.g., carbon dioxide, water, and small amounts of saturated hydrocarbons), unreacted ethylene, oxygen, and inert gases. The epoxidation reaction is generally exothermic. Thus, a coolant system 27 (e.g., a cooling jacket or a hydraulic circuit with a coolant fluid such as a heat transfer fluid or boiling water) is provided to regulate the temperature of reactor 22. The heat transfer fluid can be any of several well-known heat transfer fluids, such as tetralin (1,2,3,4-Tetrahydronaphthalene).

The gas phase promoter in reactor feed 24 is generally a compound (or compounds) that enhances the efficiency and/or activity of process 20 (FIG. 1) for producing ethylene oxide. Preferred gas phase promoters include organic chlorides. More preferably, the gas phase promoter is at least one organic chloride selected from the group consisting of methyl chloride, ethyl chloride, ethylene dichloride, vinyl chloride, and mixtures thereof. Ethyl chloride and ethylene dichloride are most preferred as the make-up organic chloride in gas phase promoter make-up feed 33. Using chlorohydrocarbon gas phase promoters as an example, it is believed that the ability of the promoter to enhance the performance (e.g., efficiency and/or activity) of process 20 for the production of ethylene oxide depends on the extent to which the gas phase promoter chlorinates the surface of the catalyst in reactor 22, for example, by depositing particular chlorine species such as atomic chlorine or chloride ions on the catalyst. However, hydrocarbons lacking chlorine atoms are believed to strip chlorides from the catalyst, and therefore, detract from the overall performance enhancement provided by the gas phase promoter. Discussions of this phenomenon can be found in Berty, “Inhibitor Action of Chlorinated Hydrocarbons in the Oxidation of Ethylene to Ethylene Oxide,” Chemical Engineering Communications, Vol. 82 (1989) at 229-232 and Berty, “Ethylene Oxide Synthesis,” Applied Industrial Catalysis, Vol. 1 (1983) at 207-238. Paraffinic compounds, such as ethane or propane, are believed to be especially effective at stripping chlorides from the catalyst. Olefins such as ethylene and propylene, are also believed to act to strip chlorides from the catalyst. Some of these hydrocarbons may also be introduced as impurities in the ethylene make-up feed 36 and/or ballast gas feed 32 or may be present for other reasons (such as the use of recycle stream 30). Typically, the preferred concentration of ethane in the reactor feed gas inlet stream 24, when present, is from 0 to about 2 mole percent.

In preferred examples, only a single species of make-up organic chloride is supplied in gas phase promoter make-up feed 33. Although the gaseous chlorine-containing promoter may be supplied as a single species, upon contact with the catalyst, other species may be formed leading to a mixture in the gas phase. Consequently, if the reaction gases are recycled such as via recycle stream 30, a mixture of species will be found in the inlet 24 of the reactor 22. In particular, the recycled reaction gases at the inlet 24 may contain ethyl chloride, vinyl chloride, ethylene dichloride and methyl chloride, even though only ethyl chloride or ethylene dichloride is supplied to the system.

As mentioned previously, it is believed that upon restart, high-efficiency ethylene oxide processes using organic chloride gas phase promoters may experience excessive release of chlorides from the surface of the catalyst and also the migration of subsurface chlorides to the surface of the catalyst. It has been found that one useful measure of this phenomenon is a relative scaled VCI Feed Gas Concentration Value, which is defined as follows:

$${C}_{VCl RS} = [ {C}_{in VCl} / {F}_{Et = MU} ] [ {F}_{Et = MU SS} / {C}_{in VCl SS} ] \qquad ( 4 )$$

wherein, C_{VCI RS}=relative scaled vinyl chloride reactor feed gas concentration (dimensionless);
C_{in VCI}=actual reactor feed gas inlet concentration of vinyl chloride (ppm molar);
F_{Et=MU}=actual make-up feed rate of ethylene (moles/hour);
F_{Et=MU SS}=pre-shutdown, steady-state make-up feed rate of ethylene (moles/hour); and
C_{in VCI SS}=pre-shutdown, steady-state reactor feed gas concentration of vinyl chloride (ppm molar)

The variables in Equation (4) may also be expressed on a mass or volumetric basis. In preferred examples, C_{in VCI} is measured by an analyzer in the reactor feed gas inlet 24, F_{Et=MU} is a real-time measurement of the feed rate of make-up ethylene, C_{in VCI SS} is a measurement of the last steady-state vinyl chloride concentration in the feed gas prior to the trip or shutdown from which the process is being restarted, and preferably, is a daily average of measured vinyl chloride concentrations during a last continuous period of steady-state operations prior to the shutdown or trip, and F_{Et=MU SS} is a measurement of the last steady state make-up feed rate of ethylene prior to the trip or shutdown from which the process is being restarted, and preferably, is a daily average value of measured make-up ethylene feed rates during a last continuous period of steady-state operations prior to the shutdown or trip.

Commercial EO processes are designed to have a low single-pass conversion, and hence provide for the recycling of unreacted ethylene and oxygen. Recycle stream 30 is also used to manage reaction heat removal and to maximize EO selectivity and yield. One example of a suitable recycle system is depicted in FIG. 1. As shown in the figure, ethylene oxide absorber 38 includes a feed stream defined by reactor product stream 26 and also includes lean water feed stream 42. Ethylene oxide absorber 38 produces a rich water stream 44 and an overhead gas stream 35 that is an intermediate stream between ethylene oxide absorber 38 and carbon dioxide removal unit 21 and which comprises unreacted olefin, oxygen, byproduct carbon dioxide, ballast gas such as methane or nitrogen, impurities such as ethane and argon. Carbon dioxide is removed in CO₂ removal unit 21 (e.g., a CO₂ scrubber coupled with a regenerator) and exits CO₂ removal unit 21 in carbon dioxide stream 40. The overhead stream 39 from CO₂ removal unit 21 is combined with CO₂ removal unit 21 bypass stream 46 to define recycle stream 30. Purge line 41 is also provided to provide for the removal of saturated hydrocarbon impurities (e.g., ethane), inerts (such as argon), and/or byproducts (as well as carbon dioxide) to prevent their accumulation in reactor feed 24. CO₂ removal unit 21 feed stream 37 is defined by ethylene oxide absorber 38 overhead stream 35, after accounting for CO₂ removal unit 21 bypass stream 46, if present, and purge line 41.

Oxygen make-up feed 34 may comprise substantially pure oxygen or air. Generally, the oxygen concentration in reactor feed gas inlet stream 24 (C_{in O2}) will be at least about 1 mole percent and preferably at least about 2 mole percent. The oxygen reactor feed gas concentration C_{in O2} will generally be no more than about 15 mole percent and preferably no more than about twelve (12) mole percent. The ballast gas 32 (e.g., nitrogen or methane) is generally from about 50 mole percent to about 80 mole percent of the total composition of reactor feed gas inlet 24.

The concentration of ethylene in reactor feed gas inlet stream 24 (C_{in Et=}) may be at least about 18 mole percent and more preferably at least about 20 mole percent. The concentration of ethylene in reactor feed gas inlet stream 24 is preferably no greater than about 50 mole percent, and more preferably is no greater than about 40 mole percent.

When present, the carbon dioxide concentration in reactor feed gas inlet stream 24 has an adverse effect on the selectivity, activity and/or stability of catalysts used in reactor 22.

Carbon dioxide is produced as a reaction by-product and may also be introduced with other inlet reaction gases as an impurity. In commercial ethylene epoxidation processes, at least a part of the carbon dioxide is removed continuously in order to control its concentration to an acceptable level in the cycle. The carbon dioxide concentration in reactor feed 24 is generally no more than about 8 mole percent, preferably no more than about 4 mole percent, and even more preferably no more than about 2 mole percent of the total composition of reactor feed gas stream 24. Water may also be present in the reactor feed gas stream 24 in a concentration that is up to 2 mole percent.

In an embodiment, the preferred concentration of ethane in the reactor feed gas inlet stream 24, when present, is up to about 2 mole percent and may reach concentrations lower than 0.1 mole percent or even 0.05 mole percent.

In accordance with a first embodiment of the present disclosure, process 20 has shut down or tripped and is off-line for a period of at least about one hour, preferably at least about two hours, and more preferably, at least about four hours. A target value of an ethylene oxide production parameter (EO_{PP T}) is selected. To begin the restart process, the ethylene concentration in the reactor feed gas inlet stream is set to an initial ethylene restart concentration value that is preferably about equal to the pre-shutdown, steady-state feed gas ethylene concentration value, which is preferably from about 20 to about 40 mole percent. The reaction temperature (T_{Rx}) is set at an initial restart reaction temperature value that is no more than five (5), preferably no more than three (3), and more preferably no more than two (2) degrees Celsius below the initial start-up steady-state reaction temperature value when the batch of catalyst was fresh (T_{Rx i SS}). At lower temperatures, the variation in selectivity with temperature can become more severe and the process often shows less stable selectivity values. At the same time, the initial restart reaction temperature value is no more than five (5), preferably no more than three (3), and more preferably, no more than two (2) degrees Celsius above the initial start-up steady-state reaction temperature value when the batch of catalyst was fresh (T_{Rx i SS}). T_{Rx i SS} is selected or empirically determined in accordance with any startup method for fresh high efficiency, Re-promoted silver catalysts known in the prior art. The initial restart reaction temperature is also preferably no greater than the pre-shutdown steady-state reaction temperature (T_{Rx SS}). The inventive method is particularly useful when T_{Rx SS} is more than five (5), more than ten (10), or more than fifteen (15) degrees Celsius above T_{Rx i SS}.

The make-up feed rate of oxygen (F_{O2 MU}) is set to an initial restart value of no more than about forty (40), preferably no more than about thirty (30), and still more preferably no more than about twenty (20) percent of the pre-shutdown, steady-state value of the make-up feed rate of oxygen (F_{O2 MU SS}). In accordance with the first embodiment, if the selectivity is less than the last steady-state value of the selectivity prior to the trip and/or shutdown (Sₛₛ) by more than three (3) percentage points, preferably by more than two (2) percentage points, and more preferably by more than 1 (one) percentage point, and if the selectivity is also trending downward (e.g., dS/dt<0, where dS/dt is preferably an averaged value over several time periods—which are preferably no longer than one hour each, more preferably no longer than 30 minutes each, still more preferably no longer than 15 minutes each and which are also preferably no less than five (5) minutes each and more preferably no less than ten (10) minutes each)—the reaction temperature (T_{Rx}) is held at whatever its current value is. Otherwise, it is increased, and, when the value is no less than the value of the initial start-up steady-state reaction temperature (T_{Rx i SS}), it is increased by no more than a maximum ramp rate of about 0.6° C., preferably 0.4° C., and more preferably 0.2° C. over any given 15 minute interval. At the same time, the minimum ramp rate is at least about 0.05° C. preferably at least about 0.1° C., and more preferably at least about 0.15° C. over any given 15 minute interval.

If the ethylene oxide production parameter (EO_{PP}) has not yet achieved its target value (EO_{PP T}) and the reaction temperature has not yet reached a value that is at least five (5) degrees Celsius above, preferably at least three (3) degrees Celsius above, and more preferably equal to its pre-shutdown steady state value (T_{Rx SS}), the ramping of reaction temperature continues. Otherwise, the temperature ramping is halted, and the method ends. As described below, sequentially or, preferably, in parallel with temperature, other selected parameters may also be increased during the restart towards their respective pre-shutdown steady-state values until either the ethylene oxide production parameter (EO_{PP}) reaches its target value (EO_{PP T}) or the other parameters also reach their respective limits based on their pre-shutdown steady-state values specified below. At this point, the restart is complete, and the process may be adjusted if necessary, using known methods to further increase the ethylene oxide production parameter to EO_{PP T} or to achieve an optimum performance at the current value of EO_{PP} according to various criteria. In one example, the feed gas make-up organic chloride feed rate and reaction temperature are adjusted to achieve maximum selectivity at the selected target value of an ethylene oxide production parameter. In another example, the feed gas make-up organic chloride feed rate F_{RCI MC} is adjusted to achieve maximum selectivity at the current value of the reaction temperature (T_{Rx}).

In accordance with a second embodiment, upon restart, process 20 has shut down or tripped and is off-line for a period of at least one hour, preferably at least two hours, and more preferably, at least about four hours. A target value of an ethylene oxide production parameter (EO_{PP T}) is selected. To begin the restart process, the ethylene concentration in the feed gas is set to an initial ethylene restart concentration value that is preferably about equal to the pre-shutdown, steady-state feed gas ethylene concentration value, which is preferably from about 25 to about 35 mole percent. The initial restart reaction temperature value is no more than five (5), preferably no more than three (3), and more preferably no more than two (2) degrees Celsius below the initial start-up steady-state reaction temperature value when the batch of catalyst was fresh (T_{Rx i SS}). At the same time, the initial restart reaction temperature value is no more than five (5), preferably no more than three (3), and more preferably, no more than two (2) degrees Celsius above the initial start-up steady-state reaction temperature value when the batch of catalyst was fresh (T_{Rx i SS}). In preferred examples, the initial restart reaction temperature value is also no greater than the pre-shutdown, steady-state reaction temperature (T_{Rx SS}). The inventive method is particularly useful when T_{Rx SS} is more than five (5), more than ten (10), or more than fifteen (15) degrees Celsius above T_{Rx i SS}.

The make-up feed rate of oxygen (F_{O2 MU}) is set to no more than about forty (40), preferably no more than about thirty (30), and still more preferably no more than about twenty (20) percent of the make-up feed rate of oxygen during the last continuous period of steady-state operation prior to the shutdown and/or trip (F_{O2 MU SS}). No make-up organic chloride is fed to the process unless and until the reactor outlet gas concentration of oxygen (C_{out O2}) reaches a value of at least about 0.5 mole percent, preferably at least about 0.7 mole percent, and still more preferably, at least about 0.9 mole percent. After the reactor outlet oxygen concentration reaches the desired value, the feed rate of the make-up organic chloride is initiated at a selected value that is no less than the minimum controllable feed rate of make-up organic chloride and no more than 30 percent, preferably no more than about 25 percent, and more preferably no more than 20 percent of the pre-shutdown, steady-state make-up organic chloride feed rate (F_{RCI MU SS}). The feed rate of the make-up organic chloride is then increased if the ratio of the values of the feed rate of the make-up organic chloride to the feed rate of make-up oxygen (i.e., F_{RCI MU}/F_{O2 MU}) is less than 115 percent of, preferably less than 105 percent of, and more preferably less than 100 percent of the ratio of the pre-shutdown, steady-state feed rate of the make-up organic chloride (F_{RCI MU SS}) to the pre-shutdown, steady-state feed rate of make-up oxygen (F_{O2 MU SS}).

If the value of the relative scaled VCI feed gas concentration (C_{VCI RS}) exceeds a value of 110 percent, preferably 105 percent, and more preferably 100 percent, the feed rate of make-up organic chloride is not increased. If (i) the selectivity is less than the last steady-state value of the selectivity prior to the trip and/or shutdown (Sₛₛ) by more than three (3) percentage points, preferably by more than two (2) percentage points, and more preferably by more than 1 (one) percentage point, (ii) the selectivity is trending downward (e.g., dS/dt<0 using an averaged value of dS/dt over several time periods, which are preferably no longer than one hour each, more preferably no longer than 30 minutes each, still more preferably no longer than 15 minutes each and which are also preferably no less than five (5) minutes each and more preferably no less than ten (10) minutes each), (iii) the relative scaled vinyl chloride feed gas concentration (C_{VCI RS}) is more than 110 percent, preferably more than 105 percent, and more preferably more than 100 percent, and (iv) C_{VCI RS} is not trending downward, the feed rate of the make-up organic chloride is decreased and preferably halted.

Once the ethylene oxide production parameter (EO_{PP}) has achieved its target value (EO_{PP T}), or the ratio of the feed rate of the make-up organic chloride to the feed rate of make-up oxygen (i.e., F_{RCI MU}/F_{O2 MU}) reaches a value that is at least 115 percent of, preferably at least 105 percent of, and more preferably at least 100 percent of the ratio of the pre-shutdown, steady-state feed rate of the make-up organic chloride to the pre-shutdown, steady-state feed rate of make-up oxygen (F_{RCI MU SS}/F_{O2 MU SS}), the feed rate of make-up organic chloride and/or feed rate of make-up oxygen are adjusted in a coordinated fashion to maintain the current ratio of the feed rates and the method ends. In parallel with the ratio of the feed rate of the make-up organic chloride to the feed rate of make-up oxygen, other selected parameters may also be increased during the restart towards their respective pre-shutdown steady-state values until either the ethylene oxide production parameter (EO_{PP}) reaches its target value (EO_{PP T}) or the other parameters also reach their respective limits based on their pre-shutdown steady-state values as described herein. At this point, the restart is complete, and the process may be adjusted if necessary, using known methods to further increase the ethylene oxide production parameter to EO_{PP} r or to achieve an optimum performance at the current value of EO_{PP} according to various criteria. In one example, the feed rate of make-up organic chloride and reaction temperature are adjusted to achieve maximum selectivity at a given value of an ethylene oxide production parameter. In another example, the make-up organic chloride feed rate is adjusted to achieve maximum selectivity at the current value of the reaction temperature (T_{Rx}).

In a third embodiment, process 20 has shut down or tripped and is off-line for a period of at least about one hour, preferably at least about two hours, and more preferably, at least about four hours. A target value of an ethylene oxide production parameter (EO_{PP T}) is selected. To begin the restart process, the ethylene concentration in the feed gas is set to an initial ethylene restart concentration value that is preferably about equal to the pre-shutdown, steady-state feed gas ethylene concentration value, which is preferably from about 25 to about 35 mole percent. The initial restart reaction temperature value is no more than five (5), preferably no more than three (3), and more preferably no more than two (2) degrees Celsius below the initial start-up steady-state reaction temperature value when the batch of catalyst was fresh (T_{Rx i SS}). At the same time, the initial restart reaction temperature value is no more than five (5), preferably no more than three (3), and more preferably, no more than two (2) degrees Celsius above the initial start-up steady-state reaction temperature value when the batch of catalyst was fresh (T_{Rx i SS}). The initial restart reaction temperature value is also preferably no greater than the pre-shutdown, steady-state reaction temperature (T_{Rx SS}). The inventive method is particularly useful when T_{Rx SS} is more than five (5), more than ten (10), or more than fifteen (15) degrees Celsius above T_{Rx i SS}.

In accordance with the embodiment, the make-up feed rate of oxygen is set to no more than about forty (40), preferably no more than about thirty (30), and still more preferably no more than about twenty (20) percent of the make-up feed rate of oxygen during the last continuous period of steady-state operation prior to the shutdown and/or trip (F_{O2 MU SS}). The make-up feed rate of oxygen (F_{O2 MU}) is then increased such that the reactor feed gas inlet stream oxygen concentration (C_{in O2}) increases by an amount that is no greater than 0.5 mole percent, preferably no greater than 0.3 mole percent, and more preferably no greater than 0.2 mole percent, averaged during any given fifteen (15) minute period. In one example, the make-up oxygen feed rate is then increased until (i) reaching a value that is at least 110 percent of, preferably at least 105 percent of, and more preferably at least 100 percent of its pre-shutdown steady-state value (F_{O2 MU} ss), (ii) the reactor inlet oxygen concentration (C_{in O2}) reaches a flammability limit (C_{in O2 flamm}), or (iii) the ethylene oxide production parameter reaches its target value, whichever comes first.

In accordance with the embodiment, if (i) the selectivity is less than the last steady-state value of the selectivity prior to the trip and/or shutdown (Sₛₛ) by more than three (3) percentage points, preferably by more than two (2) percentage points, and more preferably by more than 1 (one) percentage point, and (ii) the selectivity is also trending downward (e.g., dS/dt<0 using an averaged value of dS/dt over several time periods which are preferably no longer than one hour each, more preferably no longer than 30 minutes each, still more preferably no longer than 15 minutes each and which are also preferably no less than five (5) minutes each and more preferably no less than ten (10) minutes each), the make-up feed rate of oxygen is not increased. Once the ethylene oxide production parameter (EO_{PP}) has achieved its target value (EO_{PP T}), or the feed rate of make-up oxygen (F_{O2 MU}) has reached 110 percent of, preferably 105 percent of, more preferably 100 percent of F_{O2 MU SS}, the make-up feed rate of oxygen is held there, and the method ends. In parallel with the make-up feed rate of oxygen, other selected parameters may also be increased during the restart towards their respective pre-shutdown steady-state values until either the ethylene oxide production parameter (EO_{PP}) reaches its target value (EO_{PP T}), or the other parameters also reach their respective limits based on their pre-shutdown steady-state values as described herein. At this point, the restart is complete, and the process may be adjusted if necessary using known methods to further increase the ethylene oxide production parameter to EO_{PP T} or to achieve an optimum performance at the current value of EO_{PP} according to various criteria. In one example, the feed rate of make-up organic chloride and reaction temperature are adjusted to achieve maximum selectivity at a given value of an ethylene oxide production parameter. In another example, the make-up organic chloride feed rate is adjusted to achieve maximum selectivity at the current value of the reaction temperature (T_{Rx}).

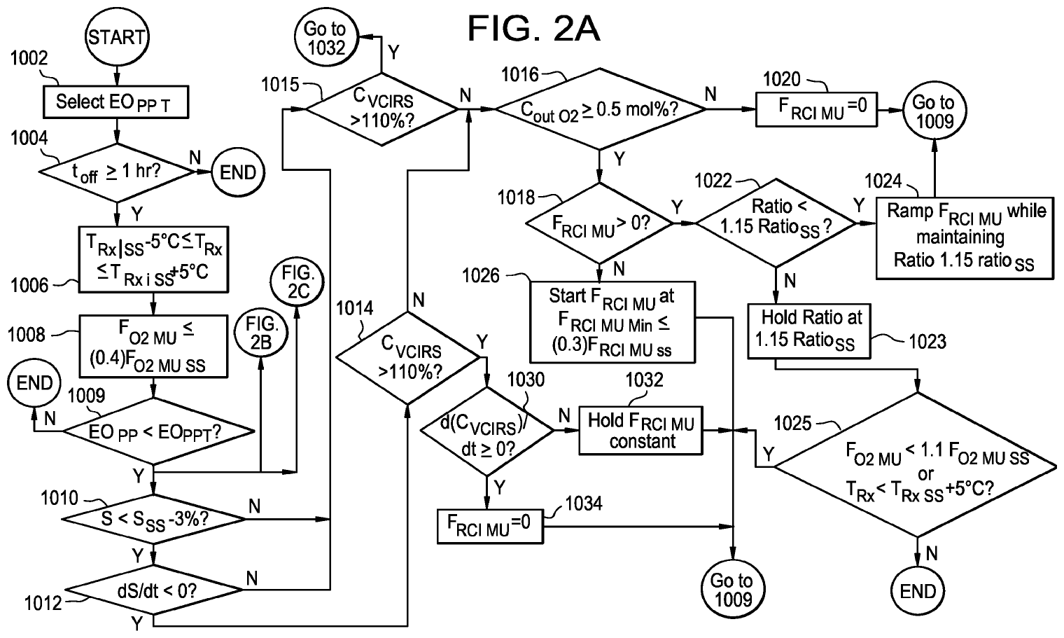

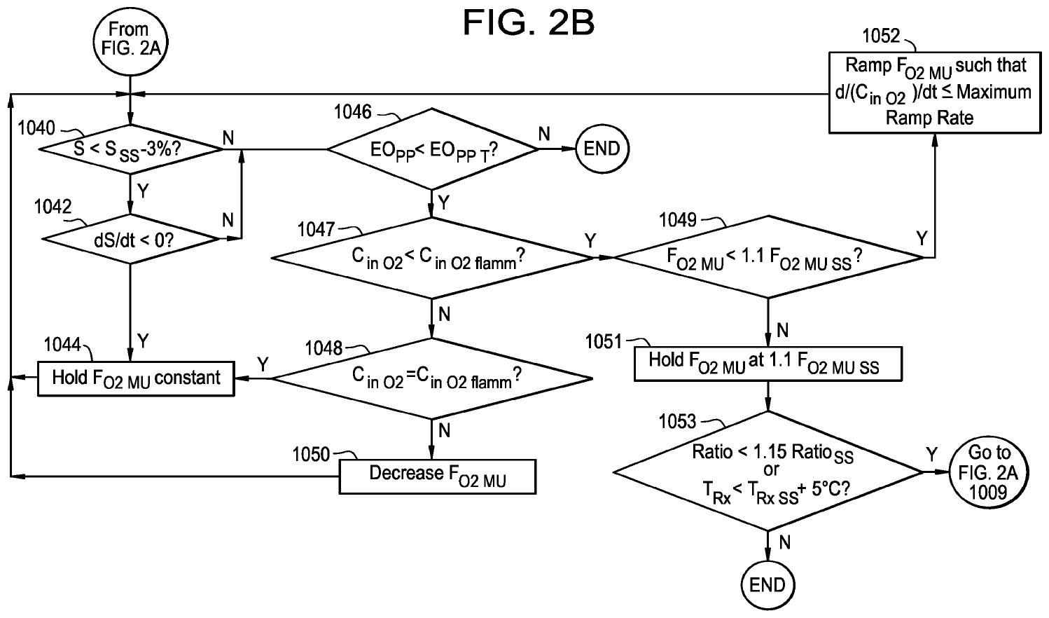

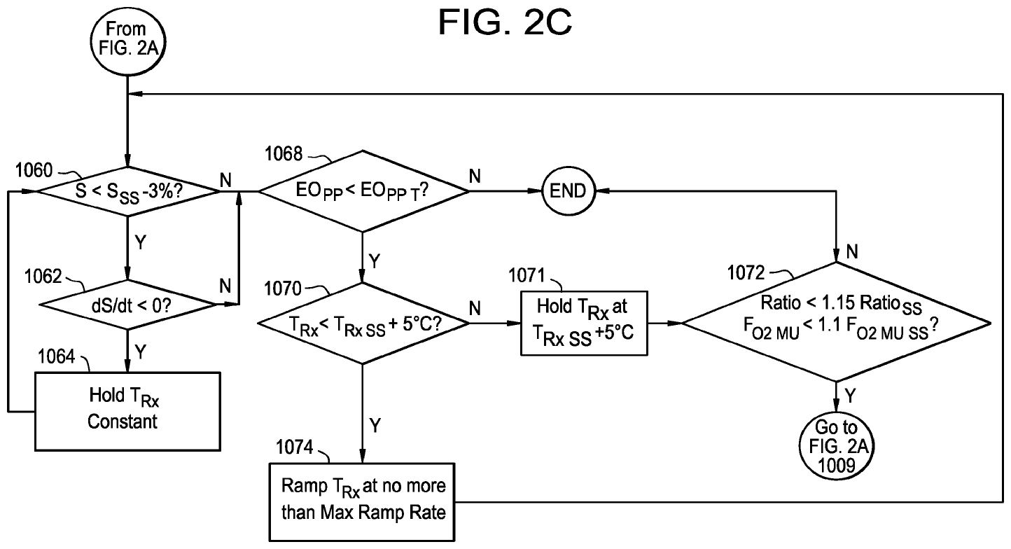

Referring to FIGS. 2A-2C a further exemplary method for improving the restart performance of a high efficiency rhenium-promoted silver catalyst is described. FIG. 2A concerns adjustments made to the feed rate of make-up organic chloride to improve restart performance. FIG. 2B concerns adjustments made to the feed rate of make-up oxygen to improve restart performance, and FIG. 2C concerns adjustments to the reaction temperature. The methods of FIGS. 2A-2C may be carried out individually or sequentially, after reaching or returning to step 1009, but are preferably carried out in parallel with one another. Each individual method includes steps 1002, 1004, 1006, 1008, and 1009 in FIG. 2A as does the integrated method where all three methods are concurrently carried out in parallel.

In each case, the process of FIG. 1 is operated during a final period of steady-state operation prior to a process trip and/or shutdown. A target value of an ethylene oxide production parameter (EO_{PP T}) is selected in step 1002 which may be the same or different than the pre-shutdown steady-state value of the ethylene oxide production parameter (EO_{PP SS}). The off-line period is at least about one hour, preferably at least two (2) hours, and more preferably, at least about four (4) hours. At the outset of the method, from the last period of steady-state operation prior to the reactor shutdown or trip, the reactor inlet concentrations of ethylene, oxygen, and at least one organic chloride (C_{in Et=}, C_{in O2}, and C_{in RCI}, respectively), have corresponding steady state values C_{in Et=SS}, C_{in O2 SS}, and C_{in RCI SS}. Similarly, from the last period of steady-state operation prior to the reactor shutdown or trip the make-up feed rates of ethylene, oxygen, and the make-up organic chloride (F_{Et=MU}, F_{O2 MU}, and F_{RCI MU}) have corresponding steady state values of F_{Et=MU} ss, F_{O2 MU SS}, and F_{RCI MU SS}. Also, from the last period of steady-state operation prior to the reactor shutdown or trip, the ratio of the make-up organic chloride feed rate to the make-up oxygen feed rate (F_{RCI MU}/F_{O2 MU}) has a corresponding value of (F_{RCI MU SS}/F_{O2 MU SS}). Additionally, from the last period of steady-state operation prior to the reactor shutdown or trip, the reaction temperature T_{Rx} and the reactor outlet concentration of ethylene oxide C_{out EtO} have corresponding steady-state values of T_{Rx SS} and C_{out EtO SS}. Note that the reactor feed gas concentrations of the compounds are the concentrations at the reactor feed gas inlet.

In step 1004 the off-line period is checked to determine if it has been sufficiently long. If it has not, the method ends. Otherwise, control is transferred to step 1006 and the reaction temperature T_{Rx} is adjusted to an initial restart reaction temperature value that is no more than five (5), preferably no more than three (3), and more preferably no more than two (2) degrees Celsius below the initial start-up steady-state reaction temperature value when the catalyst was fresh (T_{Rx i SS}). At the same time, the initial restart reaction temperature value is adjusted to no more than five (5), preferably no more than three (3), and more preferably no more than two (2) degrees Celsius above T_{Rx i SS}. In preferred examples, the initial restart reaction temperature is no greater than the pre-shutdown, steady-state reaction temperature (T_{Rx SS}). The inventive method is particularly useful when T_{Rx SS} is more than five (5), more than ten (10), or more than fifteen (15) degrees Celsius above T_{Rx i SS}.

In step 1008 the feed rate of make-up oxygen (F_{O2 MU}) is initially set to a value that is no more than 40, preferably no more than 30, and more preferably no more than 20 (twenty) percent of the average feed rate during the last period of steady-state operation prior to the shutdown (F_{O2 MU SS}).

In step 1009 the ethylene oxide production parameter is compared to the target value EO_{PP T}. If EO_{PP T} has been reached, the method ends. Otherwise, control transfers to step 1010, and/or step 1040 (FIG. 2B), and/or step 1060 (FIG. 2C).

At this point, the methods of FIGS. 2A, 2B, and 2C diverge, but they can be and preferably are carried out concurrently in parallel with one another. Referring to FIG. 2A, in step 1010, a determination is made as to whether a current value of selectivity S is significantly below the selectivity from the last period of steady-state operation prior to the shutdown (Sₛₛ). In the example of FIG. 2A, the value of the selectivity is considered significantly below the pre-shutdown steady-state value if it is less than the value of the pre-shutdown steady-state selectivity Sₛₛ by more than three percentage points, preferably by more than two percentage points, more preferably by more than one percentage point.

If step 1010 returns a value of YES, control transfers to step 1012, and a determination is made as to whether the selectivity is trending downward with time, i.e., whether dS/dt—where dS/dt is preferably an averaged value over several time periods which are preferably no longer than one hour each, more preferably no longer than 30 minutes each, still more preferably no longer than 15 minutes each and which are also preferably no less than five (5) minutes each and more preferably no less than ten (10) minutes each)—is less than zero. If it is trending downward, control transfers to step 1014. In step 1014, if the relative scaled vinyl chloride reactor feed gas concentration (C_{VCI RS}) exceeds a value of 110 percent, preferably 105 percent, and more preferably 100 percent, control transfers to step 1030 and a determination is made as to whether the relative scaled vinyl chloride reactor feed gas concentration (C_{VCI RS}) is either flat or trending upward (d (C_{VCI RS})/dt≥0)). If Step 1030 returns a value of NO (C_{VCI RS} is high but declining), the feed rate of the make-up organic chloride is held at its current value (Step 1032). If step 1030 returns a value of YES, control transfers to step 1034 and the current feed rate of the make-up organic chloride is decreased, or, preferably, halted entirely. Control then transfers back to step 1009.

If step 1010 returns a value of NO (i.e., the selectivity is not significantly lower than the previous steady-state value Sₛₛ) or if step 1012 returns a value of NO (selectivity is significantly lower but not trending downward), control transfers to step 1015. In step 1015, if the relative scaled reactor feed gas concentration of vinyl chloride (C_{VCI RS}) exceeds a value of 110 percent, preferably 105 percent and more preferably 100 percent, control transfers to step 1032, and the feed rate of make-up organic chloride is held constant. Control then transfers back to step 1009. If either step 1014 or step 1015 returns a value of NO (C_{VCI RS} is not too high), control transfers to step 1016 to determine if the reactor outlet oxygen concentration (C_{out O2}) has reached a value of at least 0.5, preferably at least 0.7, and more preferably at least 0.9 mole percent. If it has not, no make-up organic chloride promoters are fed to process 20 (step 1020), and control transfers back to step 1009. If step 1016 returns a value of YES, control transfers to step 1018 to determine if the feed rate of the make-up organic chloride (F_{RCI MC}) is greater than zero. If step 1018 returns a value of NO, in step 1026 the feed rate of the make-up organic chloride is set to a value that is no greater than 30 percent, preferably no greater than 25 percent, and more preferably no greater than 20 percent of the pre-shutdown steady-state make-up organic chloride feed rate F_{RCI MU SS}, and control transfers back to step 1009. Otherwise, if step 1018 returns a value of YES, control transfers to step 1022 to determine if the ratio of the make-up organic chloride feed rate to the make-up oxygen feed rate (F_{RCI MC}/F_{O2 MU}), which is referred to as “Ratio” in steps 1022, 1023, 1024, 1053, and 1072, is less than 115 percent of, preferably less than 105 percent of, and more preferably less than 100 percent of the ratio of the pre-shutdown steady-state value of the make-up organic chloride feed rate to the pre-shutdown steady-state value of the make-up oxygen feed rate (F_{RCI MU SS}/F_{O2 MU SS}), referred to as “Ratio ss” in steps 1022, 1023, 1024, 1053, and 1072. If it is (i.e., step 1022 returns a value of YES), the make-up organic chloride feed rate is increased to increase the value of the ratio F_{RCI MU}/F_{O2 MU} such that it approaches but does not exceed 115 percent of, preferably 105 percent of, and more preferably 100 percent of the pre-shutdown ratio F_{RCI MU SS}/F_{O2 MU SS} (step 1024), and control transfers back to step 1009. Otherwise, if step 1022 returns a value of NO, ramping of the make-up organic chloride feed rate is stopped so that F_{RCI MU}/F_{OR MU} can be held at the value that is 115 percent of, preferably 105 percent of, and more preferably 100 percent of F_{RCI MU SS}/F_{O2 MU SS} (step 1023), and control transfers to step 1025, where a determination is made of whether increases in the values of other parameters that may be ramped during the restart process can still be carried out, specifically whether the feed rate of make-up oxygen (F_{O2 MC}) is less than 110 percent of, preferably less than 105 percent of, more preferably less than 100 percent of the pre-shutdown steady-state feed rate of make-up oxygen (F_{O2 MU SS}), or the reaction temperature (T_{Rx}) has not reached a value that is 5 degrees Celsius above, preferably 3 degrees Celsius above, more preferably equal to the pre-shutdown steady-state reaction temperature (T_{Rx SS}). If step 1025 returns a value of YES (F_{O2 MU} and/or T_{Rx} can still be ramped), control returns to step 1009, otherwise the restart process is complete, and the method terminates.

FIG. 2B illustrates an embodiment of a method of adjusting make-up feed rates of oxygen to improve restart performance. Following the completion of step 1009 in FIG. 2A, control transfers to step 1040 in FIG. 2B. In step 1040 a determination is made as to whether a current value of selectivity S is significantly below the selectivity from the last period of steady-state operation prior to the shutdown (Sₛₛ). In the example of FIG. 2B, the value of the selectivity is considered to be significantly below the pre-shutdown steady-state value if it is less than the pre-shutdown, steady-state selectivity value Sₛₛ by more than three percentage points, preferably by more than two percentage points, more preferably by more than one percentage point. If S is significantly lower than Sₛₛ, step 1040 returns a value of YES, and control transfers to step 1042. In step 1042 the trend of selectivity with respect to time is evaluated. If selectivity is trending downward, dS/dt is less than zero. The evaluation of dS/dt is preferably not based on a single instantaneous value and is preferably an average taken over several time intervals which are preferably no longer than one hour each, more preferably no longer than 30 minutes each, still more preferably no longer than 15 minutes each and which are also preferably no less than five (5) minutes each and more preferably no less than ten (10) minutes each. If selectivity is trending downward, step 1042 returns a value of YES, and the current feed rate of make-up oxygen (F_{O2 MC}) is held constant. Step 1044. Control is then transferred back to step 1040.

If either step 1040 or step 1042 returns a value of NO, i.e., if the selectivity is not significantly below the pre-shutdown steady-state value or if the selectivity is significantly lower but not trending downward, control transfers to step 1046, and it is determined whether the ethylene oxide production parameter is lower than its target value (EO_{PP T}) from step 1002 in FIG. 2A. If it is not, the method ends. Otherwise, control transfers to step 1047, and the reactor inlet oxygen concentration is compared to a flammability concentration (C_{in O2 flamm}) which is preferably the flammability limit concentration less a margin for safety. If the oxygen concentration is not below the flammability concentration (step 1047 returns NO) but is determined at step 1048 to be equal to the flammability concentration, the feed rate of make-up oxygen is held constant (step 1044) and control transfers back to step 1040. If step 1048 returns NO, the oxygen make-up feed rate is decreased (step 1050), and control again transfers back to step 1040. If step 1047 returns YES (the oxygen concentration is below the flammability concentration), control transfers to step 1049, and the feed rate of make-up oxygen is compared to the pre-shutdown, steady-state value. If the feed rate of make-up oxygen F_{O2 MU} is less than 110 percent of, preferably less than 105 percent of, and more preferably less than 100 percent of its pre-shutdown steady-state value F_{O2 MU SS}, control transfers to step 1052, and the feed rate of make-up oxygen is increased to drive the process towards the target value of the ethylene oxide production parameter such that the reactor inlet oxygen concentration increases at a rate that is no more than a maximum ramp rate. Step 1052. The maximum ramp rate amount for reactor inlet O₂ concentration is no more than 0.5, preferably no more than 0.3, and more preferably no more than 0.2 mole percent, over any 15 minute interval. Thus, step 1052 may be iterative in the sense that F_{O2 MU} is increased while monitoring C_{in O2} to make sure that the resulting increase in C_{in O2} is not greater than the maximum C_{in O2} ramp rate. Following step 1052, control returns to step 1040.

In step 1049, if the make-up oxygen feed rate is at least 110 percent of, preferably at least 105 percent of, and more preferably at least 100 percent of the pre-shutdown steady state value, control transfers to step 1051, and the feed rate of make-up oxygen is held at 110 percent of, preferably 105 percent of, more preferably 100 percent of the pre-shutdown steady-state value (F_{O2 MU SS}). Control then transfers to step 1053 where a determination is made of whether increases in the values of other parameters that may be ramped during the restart process can still be carried out, specifically whether the ratio of the make-up organic chloride feed rate to the make-up oxygen feed rate (F_{RCI MC}/F_{O2 MC}) is less than 115 percent of, preferably less than 105 percent of, and more preferably less than 100 percent of the ratio of the pre-shutdown steady-state value of the make-up organic chloride feed rate to the pre-shutdown steady-state value of the make-up oxygen feed rate (F_{RCI MU SS}/F_{O2 MU SS}), or the reaction temperature (T_{Rx}) has not reached a value that is 5 degrees Celsius above, preferably 3 degrees Celsius above, more preferably equal to the pre-shutdown steady-state reaction temperature (T_{Rx SS}). If step 1053 returns a value of YES (F_{RCI MU} and/or T_{Rx} can still be ramped), control returns to step 1009 in FIG. 2A, otherwise the restart process is complete, and the method terminates.

FIG. 2C illustrates an embodiment of a method of adjusting the reaction temperature to improve the restart performance of a high efficiency, rhenium-promoted silver ethylene oxide catalyst. Following the completion of step 1009 in FIG. 2A, control transfers to step 1060 in FIG. 2C. In step 1060 a determination is made as to whether a current value of selectivity S is significantly below the selectivity from the last period of steady-state operation prior to the shutdown (Sₛₛ). In the example of FIG. 2C, the value of the selectivity is considered to be significantly below the pre-shutdown steady-state value if it is less than the pre-shutdown, steady-state selectivity value Sₛₛ by more than three percentage points, preferably by more than two percentage points, more preferably by more than one percentage point. If S is significantly lower than Sₛₛ, step 1060 returns a value of YES, and control transfers to step 1062. In step 1062 the trend of selectivity with respect to time is evaluated to determine if selectivity is trending downward (i.e., dS/dt is less than zero). The evaluation of dS/dt is preferably not based on a single instantaneous value and is preferably an average taken over several time intervals which are preferably no longer than one hour each, more preferably no longer than 30 minutes each, still more preferably no longer than 15 minutes each and which are also preferably no less than five (5) minutes each and more preferably no less than ten (10) minutes each. If selectivity is trending downward, step 1062 returns a value of YES, and the reaction temperature T_{Rx} is held at the current value. Control is then returned to step 1060.

If either the selectivity is not significantly lower than the pre-shutdown steady state value (i.e., step 1060 returns a value of NO) or the selectivity is significantly lower but not trending downward (i.e., step 1062 returns a value of NO), control transfers to step 1068. In step 1068 a determination is made as to whether the ethylene oxide production parameter is less than the target value set in step 1002 (FIG. 2A). If it is not, the restart is complete, and the method ends. Otherwise, control transfers to step 1070. In step 1070 the reaction temperature is compared to the pre-shutdown, steady-state reaction temperature. If the reaction temperature (T_{Rx}) has not reached a value that is at least 5 degrees Celsius above, preferably at least 3 degrees Celsius above, and more preferably equal to the pre-shutdown steady-state reaction temperature (T_{Rx SS}), control transfers to step 1074, and the reaction temperature is increased, at, when the reaction temperature is no less than the initial start-up steady-state reaction temperature (T_{Rx i SS}), no more than a maximum reaction temperature ramp rate of 0.6° C., preferably 0.4° C., and more preferably 0.2° C., during any given fifteen minute interval. Control is then transferred to step 1060.

If step 1070 returns a value of NO, the reaction temperature is held at a value 5 degrees Celsius above, preferably 3 degrees Celsius above, more preferably at the pre-shutdown steady-state reaction temperature (step 1071). Control then transfers to step 1072 where a determination is made of whether increases in the values of other parameters that may be ramped during the restart process can still be carried out, specifically whether the ratio of the make-up organic chloride feed rate to the make-up oxygen feed rate (F_{RCI MU}/F_{O2 MU}) is less than 115 percent of, preferably less than 105 percent of, and more preferably less than 100 percent of the ratio of the pre-shutdown steady-state value of the make-up organic chloride feed rate to the pre-shutdown steady-state value of the make-up oxygen feed rate (F_{RCI MU SS}/F_{O2 MU SS}), or the feed rate of make-up oxygen (F_{O2 MU}) is less than 110 percent of, preferably less than 105 percent of, more preferably less than 100 percent of the pre-shutdown steady-state feed rate of make-up oxygen (F_{O2 MU SS}). If step 1072 returns a value of YES (F_{RCI MU} and/or F_{O2 MU} can still be ramped), control returns to step 1009 in FIG. 2A, otherwise the restart process is complete, and the method terminates.

### EXAMPLES

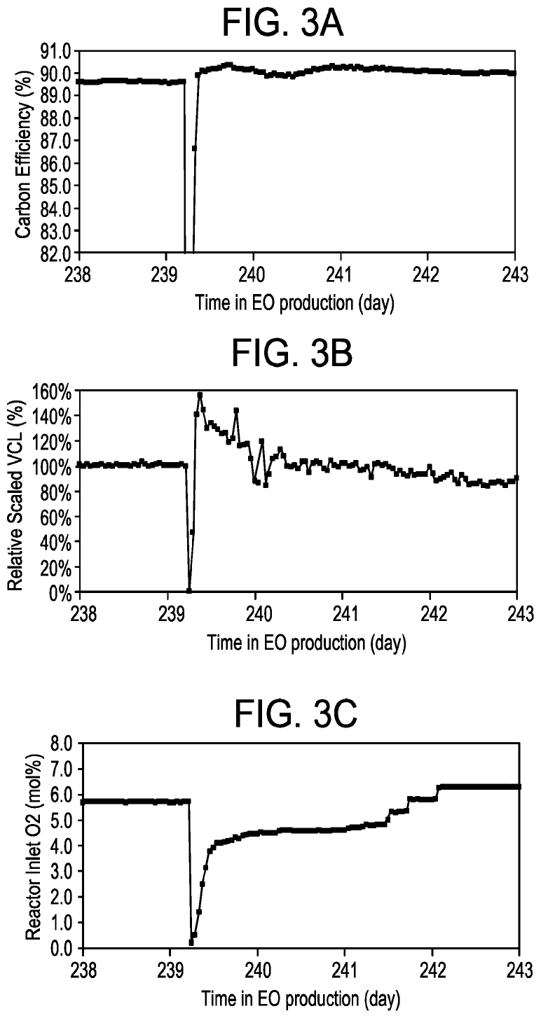

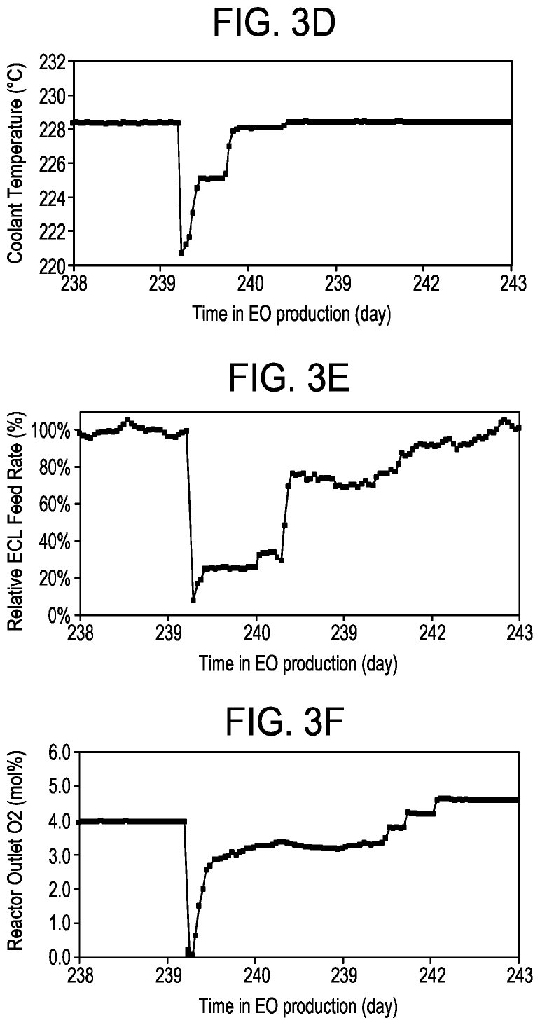

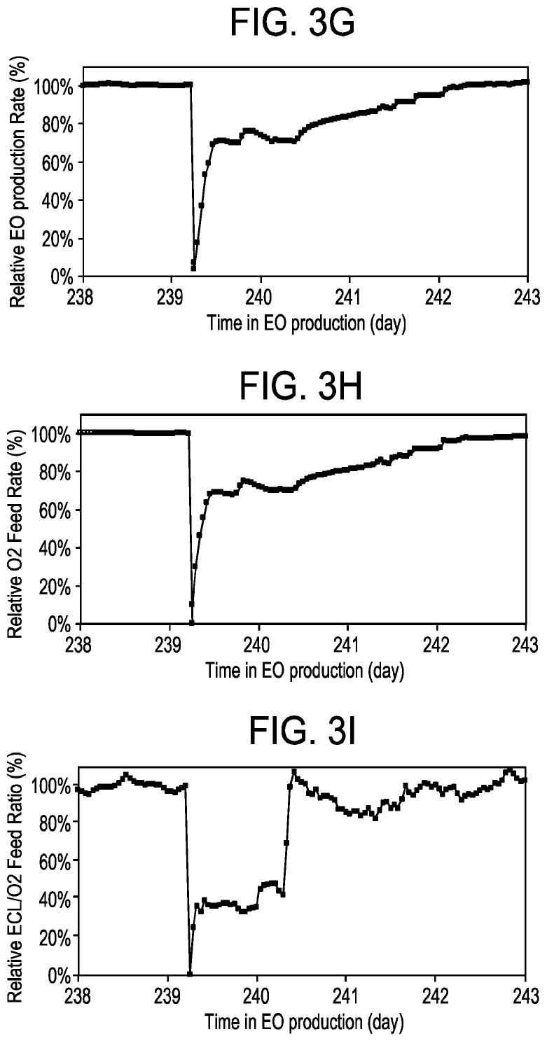

The following inventive and comparative examples are provided to illustrate the benefits of the restart methods described herein. It should be noted that the data shown in FIGS. 3A-7I is hourly averaged data and may not represent the instantaneous value of a particular parameter at the time the parameter is changed.

### Example 1

Example 1 is performed in accordance with the inventive restart methods described herein. A commercial, ethylene oxide process utilizing a high efficiency, rhenium-promoted silver ethylene oxide catalyst is subjected to a feed gas formed by combining a reactor product recycle stream with make-up streams comprising ethylene, oxygen, and ethyl chloride as a gas phase promoter. Pressure and feed gas ethylene concentration are held constant. The target value of an ethylene oxide production parameter is 100 percent of the pre-shutdown, steady-state value of the ethylene oxide production rate. The process is operated at steady state for four (4) days, and experiences a process upset of eleven (11) calendar days duration starting at about EO production day 239.2 of being on-line. The pre-shutdown, steady-state selectivity is about 89.5-89.7 percent; the pre-shutdown, steady-state reaction temperature (measured as the reactor coolant temperature) is about 228.4° C. The pre-shutdown, steady-state reactor inlet oxygen concentration is about 5.6-5.8 mole percent, and the pre-shutdown, steady-state reactor outlet oxygen concentration is about 4.0 mole percent. The initial start-up steady-state reaction temperature value (T_{Rx i SS}) is 225° C.

The process is then restarted as shown in FIGS. 3A-3I. It should be noted that the x-axis in FIGS. 3A-7I is the elapsed time in ethylene oxide production days, i.e., the elapsed time during which ethylene oxide was produced since the initial start-up of the fresh catalyst batch. As a result, the shut-down period of eleven days is not evident as it otherwise would be if the measured time were not based on a production parameter. Because the graphs show elapsed time, the numbered locations represent completed EO production days. For example, the x-axis location identified as 240 marks the completion of the 240th day, after which EO production day 241 begins.

The method begins by setting the reaction temperature (here, the reactor coolant outlet temperature) at 220° C. (FIG. 3D), which is five (5) degrees Celsius below the initial steady-state temperature after start-up on fresh catalyst (T_{Rx i SS}). Make-up oxygen is initially fed at a first volumetric flow rate that is 10 percent of the pre-shutdown volumetric flow rate (FIG. 3H). The temperature is slowly ramped up by 5° C. to reach 225° C. over a period of about 6 hours such that the ramp rate never exceeded 0.4° C. in any given 15 minute period (FIG. 3D). During this same period, the relative make-up oxygen feed rate is increased to about 70 percent, and the reactor feed gas oxygen concentration at the reactor inlet increases from 0 to about 3.8-3.9 mole percent, such that the ramp rate does not exceed 0.3 mole percent during any given 15 minute period.

Within about 1-2 hours of restart, the reactor outlet oxygen concentration reaches 0.5 mole percent (FIG. 3F), at which point make-up ethyl chloride is introduced into the reactor feed at its minimum controllable feed rate and held there for approximately 3 hours. Although the relative scaled reactor feed gas VCI concentration C_{VCI RS} shoots as high as about 155% (FIG. 3B), it falls in a roughly monotonic manner until reaching 100 percent shortly before EO production day 240.0 As indicated in FIG. 3E, the feed rate of make-up ethyl chloride is held constant while C_{VCI RS} remains above about 110 percent. Once C_{VCI RS} drops below about 100 percent, the relative make-up ethyl chloride feed rate is increased by about 5 percent. However, C_{VCI RS} spikes to 120 percent, and the feed rate of make-up ethyl chloride is held constant until C_{VCI RS} drops below 100 percent. At the beginning of the restart, the relative make-up ethyl chloride feed rate is ramped to a rate that maintains a constant ratio of the relative make-up ethyl chloride feed rate to the relative make-up oxygen feed rate (FIGS. 3E and 3I). About a day later, the relative feed rate of make-up ethyl chloride is ramped from 30 percent to 75 percent and then held at about 75 percent when the ratio of the make-up ethyl chloride feed rate to the make-up oxygen feed rate reaches at least 100 percent of the ratio of the pre-shutdown steady-state value of the make-up ethyl chloride feed rate to the pre-shutdown steady-state value of the make-up oxygen feed rate (FIG. 3I). After about another day, the relative feed rate of make-up ethyl chloride is slowly increased toward 100 percent, while at the same time C_{VCI RS} approaches a value of about 80-85 percent in a generally monotonic manner.

About three (3) hours after restart begins, the selectivity trends monotonically upward to a value of about 89.8 percent. It then reaches values that fluctuate between 89.8 percent and 90.3 percent before monotonically reaching 90.0 percent one (1) EO production day and 8 EO production hours after restart. The ethylene oxide production rate quickly increases to about 70 percent of the pre-shutdown value within the first several hours after restart and fluctuates slightly from that point until about EO production day 240.4, after which it gradually and monotonically approaches its pre-shutdown, steady-state value at about EO production day 242.3. It is believed that the initially high relative scaled VCI feed gas concentration values are indicative of the diffusion and desorption of subsurface chlorides and their subsequent redeposition on the catalyst via recycle to the reactor inlet feed gas. However, using the inventive method described herein, the process smoothly and stably returned to its pre-shutdown steady-state selectivity and EO production parameter value.

### Comparative Example 1

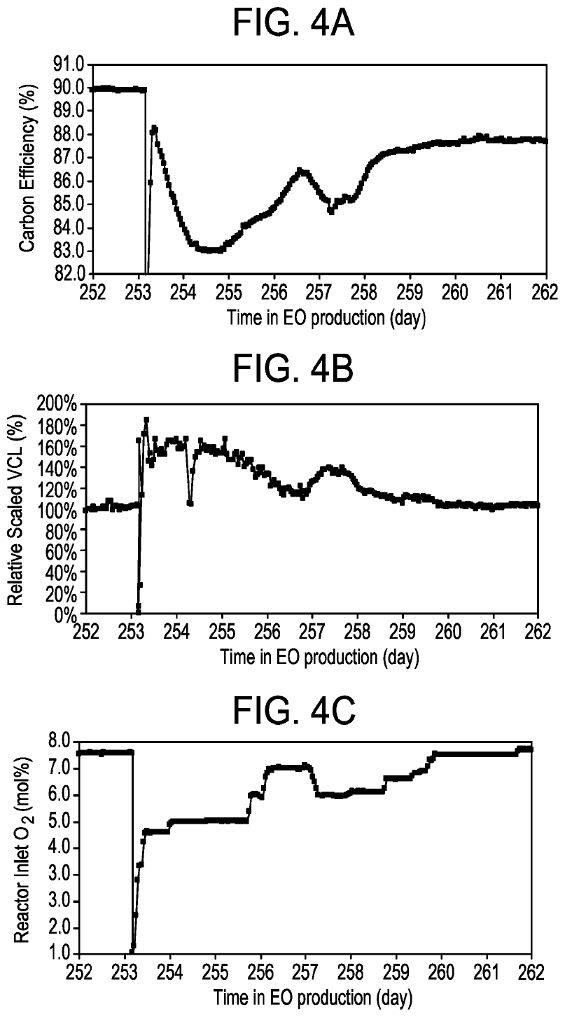

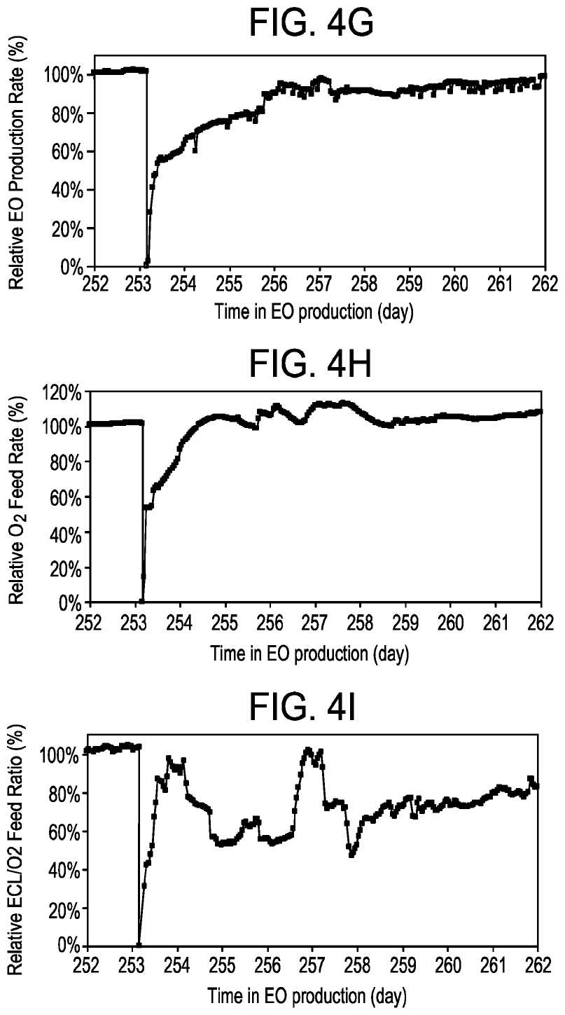

This example illustrates how poor selection of restart operating conditions can lead to a persistent loss of selectivity following restart. The process of Example 1 operates at a continuous steady-state period of at least three (3) days following the period of FIGS. 3A-3I and then experiences a process upset leading to a five (5) day shutdown at about ethylene oxide production day 253.2 with the same batch of catalyst used in Example 1. The process is restarted as shown in FIGS. 4A-4I.

To begin the restart process, the reaction temperature is brought up to slightly over 220° C., which is about five (5) degrees Celsius lower than the initial steady-state temperature after starting up on fresh catalyst (T_{Rx i SS}) (FIG. 4D). Make-up oxygen is introduced at about the same time at a relative make-up oxygen feed rate of about 14-16 percent (FIG. 4H). The reactor outlet oxygen concentration (C_{out O2}) quickly reaches 0.5 percent (FIG. 4F), and at about EO production day 253.3, make-up ethyl chloride is fed to the reactor at a relative make-up ethyl chloride feed rate that is initially about 14-16 percent (FIG. 4E). The relative feed rates of both make-up ethylene and make-up oxygen are increased over a period of about 3-4 hours, with the relative make-up oxygen feed rate being increased from about fourteen (14) percent to about 53 percent. The reactor inlet oxygen concentration jumps from 0 to 3.5 percent in that same time frame (peak ramping rate of 0.30-0.35 mole percent in 15 minutes). FIG. 4C. However, in this time frame, the selectivity peaks at a value of about 88.3 percent and then begins dropping for the remainder of day 253, while the relative scaled feed gas VCI concentration exceeds 135 percent. Despite this, the relative make-up ethyl chloride feed rate is increased approximately linearly until reaching a value of nearly 90 percent of its pre-shutdown steady-state value at about EO production day 254.2. FIG. 4E.

The selectivity reaches a minimum of about 83 percent about half-way into EO production day 254 and takes about two days to reach 86.5 percent. About nine (9) days after the restart begins, the selectivity reaches a new steady-state value of about 87.8 percent, well below the pre-shutdown value of 90.0 percent. It is believed that the excessive addition of ethyl chloride from continued ramping of ethyl chloride feed rate while the relative scaled VCI feed gas concentration remains high, and especially the ramping of temperature and feed gas make-up oxygen while the selectivity trends downward and remains significantly below its pre-shutdown, steady-state value significantly exacerbate the problem of subsurface chloride diffusion, desorption and surface re-deposition, causing the process to remain unstable and the selectivity to remain persistently below the pre-shutdown steady-state value even after the ethylene oxide production rate reaches its pre-shutdown steady-state value on about EO production day 262.

### Comparative Example 2

A pilot plant utilizing a high-efficiency ethylene oxide catalyst is subjected to a feed gas formed by combining a recycle stream with make-up feed streams of oxygen, ethylene, and ethyl chloride (as a gas phase promoter). The target value of the EO production parameter is its pre-shutdown, steady-state value. Pressure and feed gas ethylene concentration are held constant.

The process is operated at steady state for three (3) days and experiences a process trip after about 49.2 ethylene oxide production days of operation after first being on-line with fresh catalyst. The shutdown lasts five (5) days.

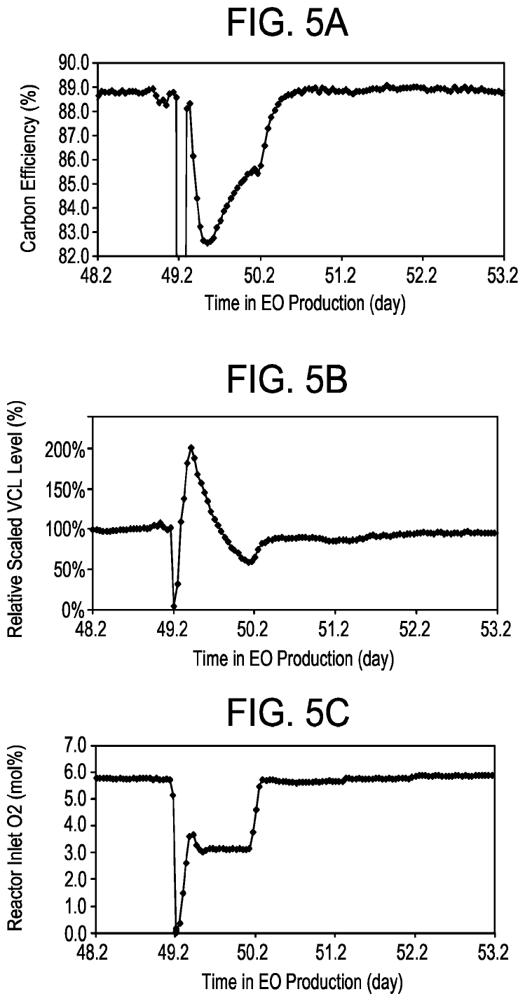

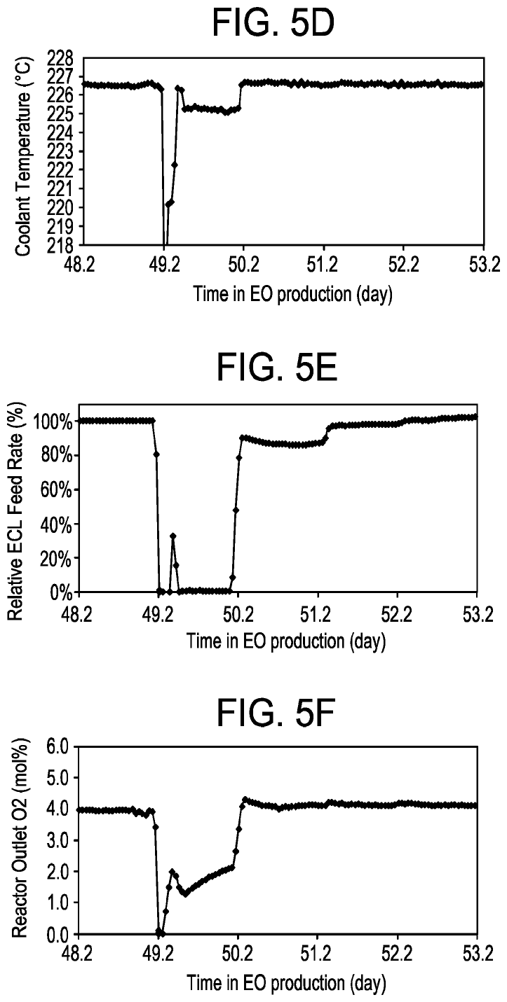

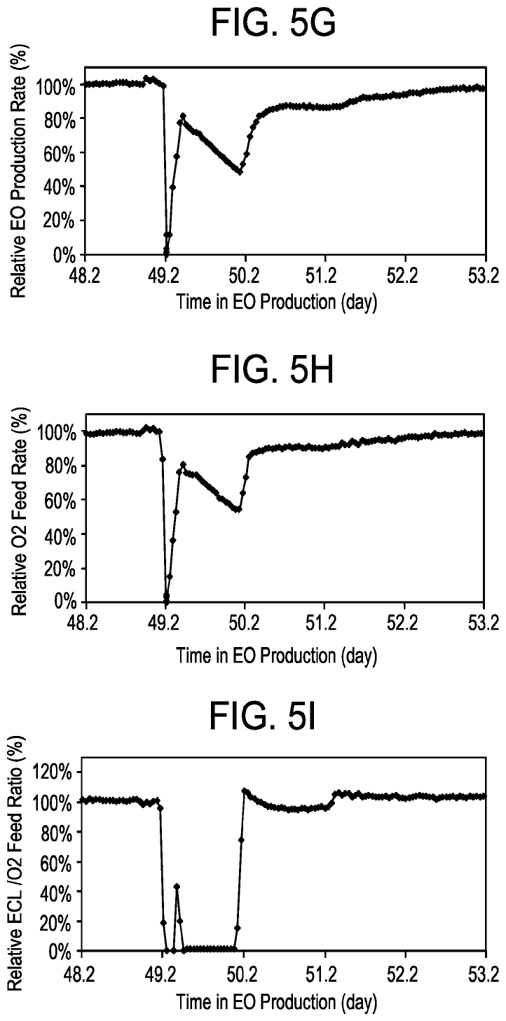

The process is restarted as shown in FIGS. 5A-5I. The process restarts with a reaction temperature (as measured by the reactor outlet coolant temperature) of about 220.1° C. (FIG. 5D), which is about 4.9 degrees Celsius lower than the initial steady-state reaction temperature (T_{Rx i SS}) following start-up on fresh catalyst. The temperature is ramped up to about 226.5° C. within the next 3 hours with a peak ramp rate of approximately one degree Celsius per 15 minutes. However, after quickly spiking to a value of about 88.3 percent, which is slightly less than the 88.5 percent, pre-shutdown steady-state selectivity, the selectivity begins trending quickly downward, reaching a value of about 82.5 percent on ethylene oxide production day 49.5. FIG. 5A.

The initial relative make-up oxygen feed rate at restart is about 15 percent (FIG. 5H) and is increased to about 80 percent by the completion of ethylene oxide production day 49.5. During this same period, the average reactor inlet oxygen concentration rises by about 3.8 mole percent, with a peak ramping rate of about 0.3 mole percent/15 minutes FIG. 5C). This ramping of oxygen feed rate and concentration occurs even while the selectivity trends sharply downward and remains below the pre-shutdown steady-state value. The reactor outlet oxygen concentration reaches 0.8 mole percent by about 49.3 ethylene oxide production days The make-up ethyl chloride feed is subsequently initiated at a relative feed rate of about 35 percent.

Following the beginning of the restart, the relative scaled feed gas VCI concentration value rises quickly to roughly 200 percent near the completion of day 49.5 (FIG. 5B) and then trends downward, falling below its pre-shutdown steady state value by the completion of ethylene oxide production day 49.8. The relative scaled VCI concentration value shows an inverse trend relative to selectivity, with the selectivity dropping quickly (FIG. 5A) as the relative scaled VCI value spikes to 200 percent and then only partially recovering as the relative scaled VCI value falls. Upon observation of the spike in the relative scaled VCI value, the make-up ethyl chloride feed is stopped at the completion of ethylene oxide production day 49.4, which eventually helps restore the selectivity to its pre-shutdown steady-state value by the completion of ethylene oxide production day 50.7 as the make-up ethyl chloride feed is not resumed until after the relative scaled VCI value has declined to well below 100%. However, the EO production parameter does not return to its pre-shutdown steady-state value by the completion of day 53.2. It is believed that the initially poor post-restart selectivity performance is due to increasing the reaction temperature and the feed rate of make-up oxygen while the selectivity is below its pre-shutdown steady-state value and trending downward, thereby prolonging and deepening the effects of the post-restart excursion in feed gas vinyl chloride concentration.

### Example 2

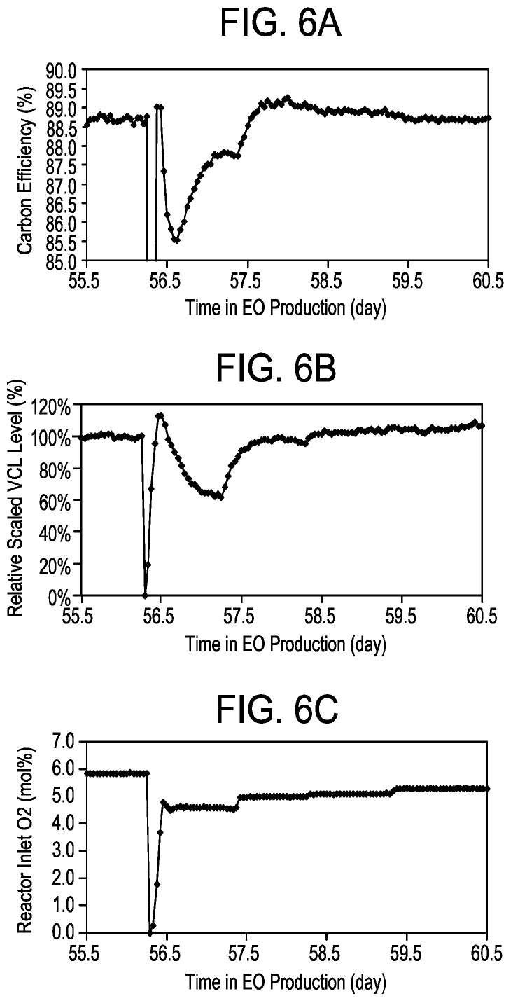

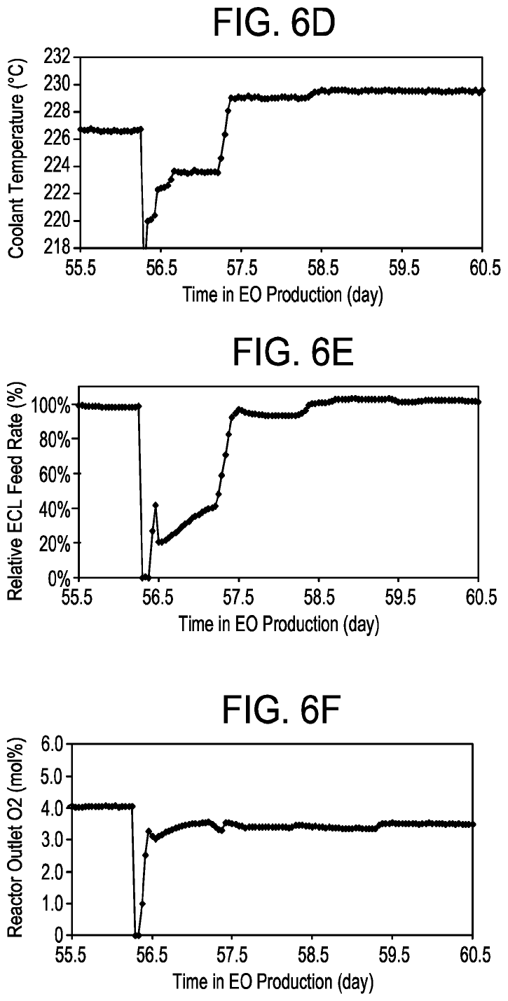

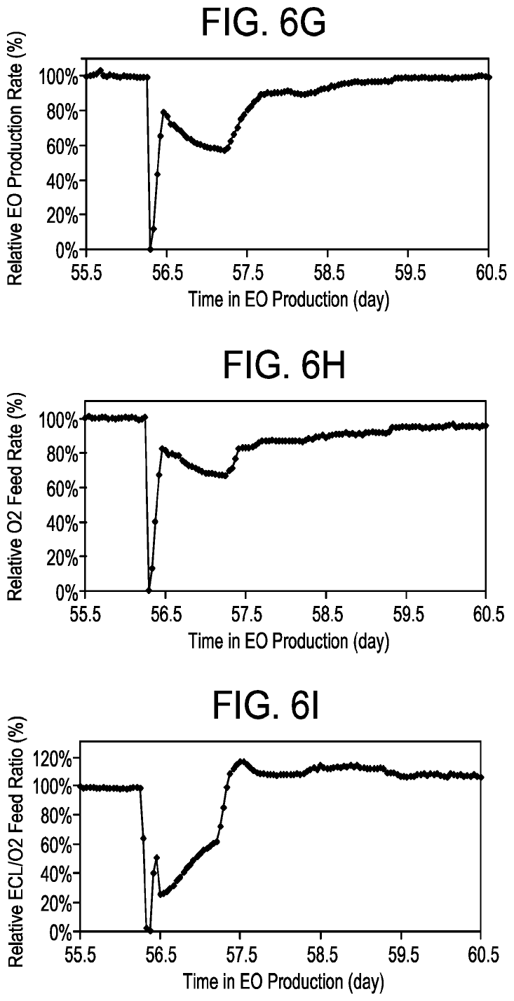

The pilot plant and catalyst of Comparative Example 2 continue to operate and then experience another process upset of about four (4) days starting at the completion of ethylene oxide production day 56.3. Upon restart, the reaction temperature (reactor coolant temperature) is 220° C. and is ramped up to about 222.3° C. in about three (3) hours. The results are shown in FIGS. 6A-6I. The EO production parameter target value is the pre-shutdown value. Pressure and feed gas ethylene concentration are held constant.

In this same time period, make-up oxygen is introduced into the feed gas at an initial relative feed rate of about thirteen (13) percent (FIG. 6H), reaching a relative make-up feed rate of about 82 percent at the completion of ethylene oxide production day 56.5. In the same time period between the completion of 56.3 to about 56.5 ethylene oxide production days, the reactor inlet oxygen concentration increases from zero to about 4.8 mole percent, with a peak ramping rate of about 0.5 mole percent per 15 minutes (FIG. 6C). Also, in this same time period, the relative make-up ethyl chloride feed rate increases from zero percent to about 42 percent (FIG. 6E), which is accompanied by the relative scaled VCI feed gas concentration increasing from zero to 113 percent. However, in response to the observation of relative scaled VCI feed gas concentration exceeding 100 percent, the relative make-up ethyl chloride feed rate is brought back down to about 21 percent and is then progressively increased to about 41 percent by the completion of ethylene oxide production day 57.2, by which point the relative scaled VCI concentration has decreased to about 64% of the pre-shutdown level. Consequently, the ramping rate for the make-up ethyl chloride feed is increased such that the relative feed rate approaches 100% of the pre-shutdown value by the completion of day 57.5, at which point the ramping of the make-up ethyl chloride feed rate is halted as the relative ratio of the make-up ethyl chloride feed rate to the make-up oxygen feed rate reaches 115 percent. Starting at the completion of ethylene oxide production day 59.3 through the completion of day 60.5, the relative scaled VCI fluctuates between about 100 and about 110 percent. During the time from the completion of ethylene oxide production day 58 to the completion of day 60.4, the relative feed rate of make-up ethyl chloride fluctuates between 93% and 103%, and the relative ratio of the make-up ethyl chloride feed rate to the make-up oxygen feed rate fluctuates between about 105 and 114 percent.

The reaction temperature is held essentially constant at about 223.6° C. from the completion of ethylene oxide production day 56.7 to the completion of day 57.2 and is then ramped up to about 229° C. over a four-hour period, with a peak ramping rate of about 0.57° C. per 15 minutes. It is held constant until the completion of ethylene oxide production day 58.3, after which it is ramped up to about 229.5° C.

The selectivity spikes to about 89.0 percent (FIG. 6A) by the completion of ethylene oxide production day 56.4 and then drops precipitously to 85.5 percent by the completion of day 56.6, slightly after the relative scaled VCI feed gas concentration peaks at about 113%. The selectivity then slowly increases to 89.0 percent by the completion of ethylene oxide production day 57.6 and slowly approaches the pre-shutdown steady-state value of 88.7 percent. After a brief upward spike, the relative ethylene oxide production rate drops (FIG. 6G) in parallel with the relative feed rate of make-up oxygen from ethylene oxide production day 56.3 to day 57.3 before making a gradual ascent to the pre-shutdown steady-state value from day 57.3 to day 59.3. It is believed that the actions taken when the relative scaled vinyl chloride concentration exceeded 100 percent contributed to the rapid recovery of the process from the precipitous selectivity decline after day 56.4.

### Comparative Example 3

A commercial ethylene oxide plant is subjected to a reactive mixture of ethylene and oxygen with an ethylene dichloride gas phase promoter for about 126 ethylene oxide production days and then shuts down at the beginning of day 127. Reactor pressure and feed gas ethylene concentration are held constant.

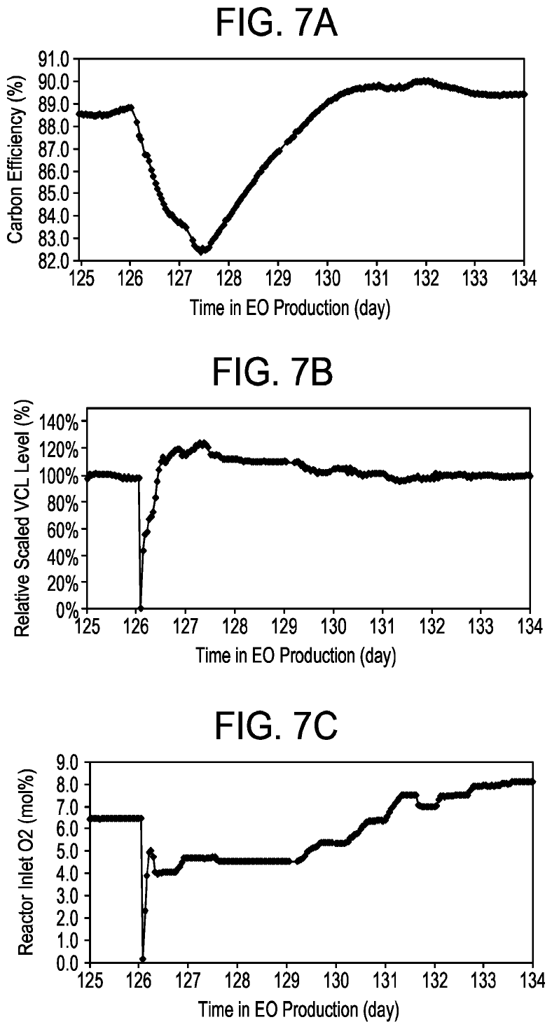

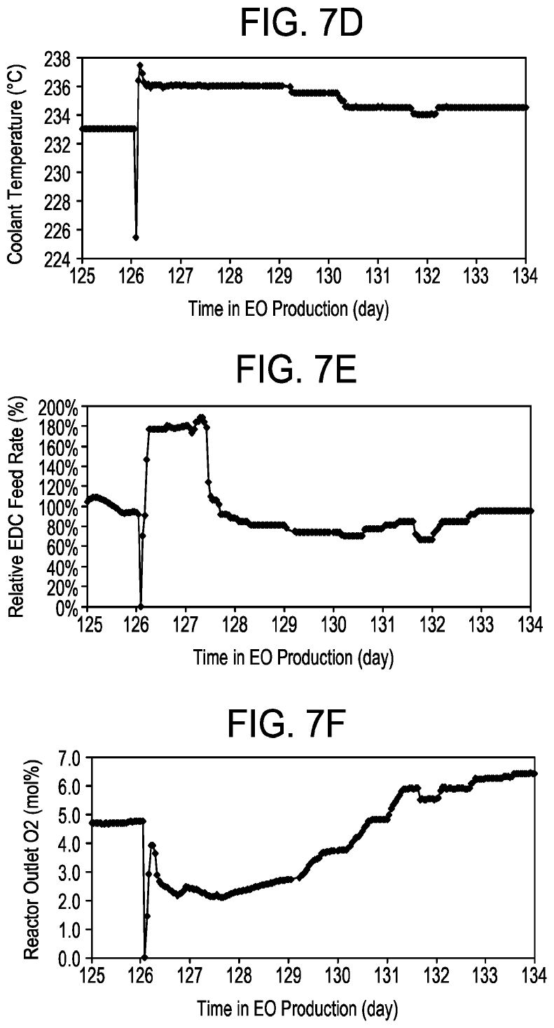

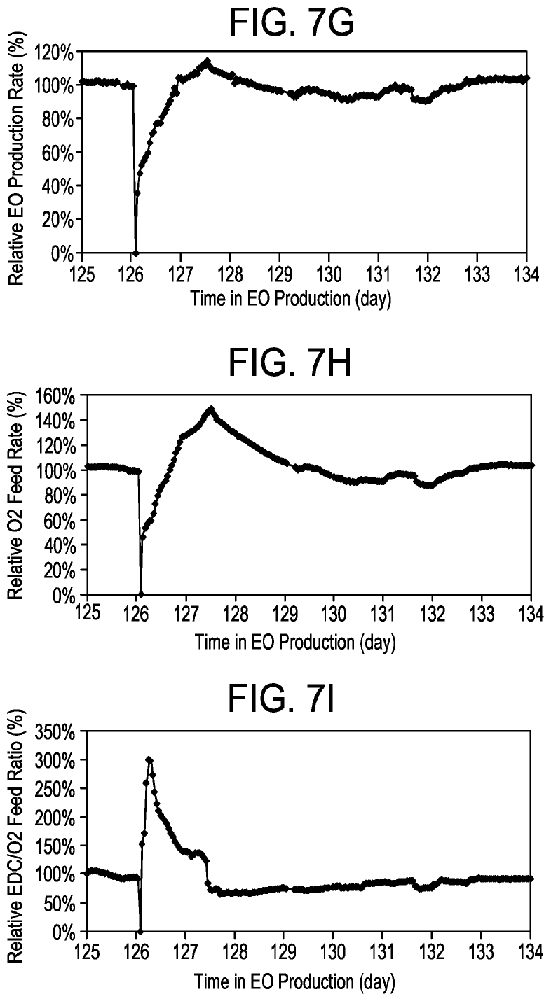

For the restart, the reaction temperature starts at 225.4° C. (slightly above the initial steady state temperature after starting up on fresh catalyst) and is quickly increased to 236.4° C. within about an hour, corresponding to a ramp rate of 2.75° C./15 minutes (FIG. 7D). The relative feed rate of make-up oxygen increases from 0 percent to about 60 percent within the first four hours of the restart (FIG. 7H). During the same period, the reactor inlet oxygen concentration rises rapidly to about 5.0 mole percent (FIG. 7F), with a peak rate of increase of 0.6 mole percent/15 minutes during the first hour. The relative feed rate of make-up ethylene dichloride (EDC) increases from 0 to about 176 percent in the same period and is then held constant for about a day after the relative, scaled VCI concentration reaches and exceeds 100 percent, eventually peaking at 123 percent before the feed rate of make-up EDC is reduced but not all the way to the minimum controllable feed rate. Throughout this initial portion of the restart, the relative ratio of the make-up ethylene dichloride feed rate to the make-up oxygen feed rate is generally well in excess of 120 percent, even exceeding 250 percent for several hours.

The pre-shutdown steady-state selectivity is about 88.7 percent. The selectivity is initially about 88.0 percent at the beginning of the restart at the beginning of ethylene oxide production day 127 before dropping precipitously and continuously to a value of 82.5 percent on about day 127.5. It then slowly recovers to reach a value of about 90 percent at the end of day 132. It is believed that the poor initial selectivity performance is attributable to increasing the reaction temperature too quickly upon restart and also feeding excessive quantities of make-up ethylene dichloride relative to make-up oxygen from the beginning of the restart until day 127.4.

1. A method for restarting an ethylene oxide production process after a period of being shutdown for at least one hour following a continuous steady state period, wherein during the continuous steady state period the process is operated by: (i) feeding a reactor feed gas to a reactor containing a batch of a high-selectivity, rhenium-promoted silver catalyst, wherein the reactor feed gas comprises ethylene at a pre-shutdown steady state ethylene concentration, oxygen at a pre-shutdown steady state oxygen concentration, and at least one organic chloride at a pre-shutdown steady state at least one organic chloride concentration, wherein the at least one organic chloride comprises vinyl chloride at a pre-shutdown steady state vinyl chloride concentration, to yield a reactor product comprising ethylene oxide at a pre-shutdown steady-state value of a selectivity of the process toward the production of ethylene oxide, wherein the ethylene oxide production process is characterized by an ethylene oxide production parameter having a pre-shutdown steady state value, and a reaction temperature has a pre-shutdown steady state reaction temperature value; (ii) recycling a portion of the reactor product to form a reactor recycle; and (iii) combining the reactor recycle with make-up streams comprising at least make-up ethylene at a pre-shutdown steady-state feed rate of make-up ethylene, make-up oxygen at a pre-shutdown steady state feed rate of make-up oxygen, and a make-up organic chloride selected from ethyl chloride and ethylene dichloride at a pre-shutdown steady state feed rate of the make-up organic chloride, to form the reactor feed gas, wherein the period of being off-line is a period during which no make-up oxygen is fed to the batch of the high-selectivity, rhenium-promoted silver catalyst, the method for restarting comprising:
again feeding a reactor feed gas to the batch of the high-selectivity, rhenium-promoted, silver catalyst, wherein during the step of again feeding a reactor feed gas, the reactor feed gas initially comprises ethylene at an initial ethylene restart concentration and the at least one organic chloride at an initial restart concentration of the at least one organic chloride, and the reactor feed gas is formed by combining the reactor recycle with make-up streams comprising at least make-up ethylene at an initial restart feed rate of make-up ethylene and make-up oxygen at an initial restart feed rate of make-up oxygen; and
adjusting the reaction temperature to an initial restart reaction temperature value that is no more than five (5) degrees Celsius above an initial start-up steady-state reaction temperature value when the high-selectivity catalyst was fresh.

2. The method of claim 1, wherein the initial restart reaction temperature value is no more than the pre-shutdown steady-state reaction temperature value.

3. A method for restarting an ethylene oxide production process according to claim 1, wherein the initial restart reaction temperature value is below the initial start-up steady-state temperature value when the high-selectivity catalyst was fresh by no more than five (5) degrees Celsius.

4. A method of for restarting an ethylene oxide production process according to claim 2, wherein during an initial start-up period prior to the continuous steady state period, the process is operated by: (i) feeding an initial start-up reactor feed gas to the reactor, wherein the initial start-up reactor feed gas comprises ethylene at an initial start-up ethylene concentration, and (ii) adjusting the reaction temperature to the initial start-up steady-state reaction temperature value.

5. The method of claim 4, wherein the pre-shutdown steady state ethylene concentration is equal to the initial ethylene restart concentration.

6. A method for restarting an ethylene oxide production process according to claim 2, wherein the method further comprises:
selecting a target value of the ethylene oxide production parameter; and
again feeding the make-up organic chloride to the batch of the high-selectivity, rhenium-promoted silver catalyst at an initial restart feed rate of the make-up organic chloride no sooner than when a concentration of oxygen in the reactor product equals or exceeds 0.5 mole percent of the reactor product, and not feeding the make-up organic chloride to the batch of the high-selectivity, rhenium-promoted silver catalyst when the concentration of oxygen in the reactor product is less than 0.5 mole percent of the reactor product.

7. The method of claim 6, wherein the initial restart feed rate of the make-up organic chloride, on a molar basis, is no more than 30 percent of the pre-shutdown steady state feed rate of the make-up organic chloride.

8. A method for restarting an ethylene oxide production process according to claim 6, wherein the method for restarting further comprises increasing the feed rate of make-up oxygen until a first one of the following occurs: (i) the feed rate of make-up oxygen reaches at least 110 percent of the pre-shutdown steady state feed rate of make-up oxygen; (ii) a reactor feed gas oxygen concentration reaches an oxygen flammability concentration, and (iii) the ethylene oxide production parameter reaches a value no less than the selected target value of the ethylene oxide production parameter.

9. The method of claim 8, wherein during the step of increasing the feed rate of make-up oxygen, during any 15 minute period, the reactor feed gas oxygen concentration does not increase by more than 0.5 mole percent.

10. A method for restarting an ethylene oxide production process according to claim 8, wherein the step of increasing the feed rate of make-up oxygen is not carried out if the selectivity is less than the pre-shutdown steady-state value of the selectivity by more than three (3) percentage points, and the selectivity is trending downward.

11. A method for restarting an ethylene oxide production process according to claim 6, wherein the method for restarting further comprises increasing the feed rate of the make-up organic chloride if and only if a relative scaled vinyl chloride reactor feed gas concentration is no greater than 110 percent.

12. The method of claim 11, wherein the step of increasing the feed rate of the make-up organic chloride is carried out until a first one of the following occurs: (i) a ratio of the feed rate of the make-up organic chloride to the feed rate of make-up oxygen reaches at least 115 percent of a ratio of the pre-shutdown steady-state feed rate of the make-up organic chloride to the pre-shutdown steady-state feed rate of make-up oxygen, and (ii) the ethylene oxide production parameter reaches a value no less than the selected target value of the ethylene oxide production parameter.

13. The method of claim 11, wherein the feed rate of the make-up organic chloride is stopped if the relative scaled vinyl chloride feed gas concentration is greater than 110 percent and not trending downward, the selectivity is less than the pre-shutdown steady-state value of the selectivity by more than three (3) percentage points, and the selectivity is trending downward.

14. A method of restarting an ethylene oxide production process according to claim 11, wherein the feed rate of the make-up organic chloride is not increased if the relative scaled vinyl chloride feed gas concentration is greater than 110 percent and trending downward, the selectivity is less than the pre-shutdown steady-state value of the selectivity by more than three (3) percentage points, and the selectivity is trending downward.

15. A method for restarting an ethylene oxide production process according to claim 1, wherein during the step of again feeding a reactor feed gas, the reactor feed gas initially does not comprise oxygen.

16. A method for restarting an ethylene oxide production process according to claim 6, wherein the method for restarting further comprises increasing the reaction temperature until a first one of the following occurs: (i) the ethylene oxide production parameter reaches a value no less than the selected target value of the ethylene oxide production parameter and (ii) the reaction temperature reaches a value higher than the pre-shutdown steady state reaction temperature value by no less than five (5) degrees Celsius.

17. The method of claim 16, wherein during the step of increasing the reaction temperature, during any 15-minute period, the reaction temperature does not increase by more than 0.6° C., when the value of the reaction temperature is no less than the value of the initial start-up steady-state reaction temperature.

18. A method for restarting an ethylene oxide production process in accordance with claim 16, wherein the step of increasing the reaction temperature is not carried out if the selectivity is trending downward and the selectivity is less than the pre-shutdown steady-state value of the selectivity by more than three (3) percentage points.

19. The method of claim 1, wherein the pre-shutdown steady state reaction temperature value is more than five (5) degrees Celsius higher than the initial start-up steady-state reaction temperature value.

20. The method of claim 1, wherein the pre-shutdown steady state ethylene concentration is from 25 mole percent to 35 mole percent of the reactor feed gas.

21.-22. (canceled)
